(19) United States
(12) Patent Application Publication
(10) Pub. No.: US 2025/0288561 A1
(43) Pub. Date: Sep. 18, 2025

(54) NEZULCITINIB FOR DELIVERY BY NEBULIZED ORAL INHALATION

(71) Applicant: THERAVANCE BIOPHARMA R&D IP, LLC, South San Francisco, CA (US)

(72) Inventors: Nathan David PFEIFER, Montara, CA (US); David L. Bourdet, Millbrae, CA (US); Edmund J. Moran, San Francisco, CA (US); Rajeev Saggar, Paradise Valley, AZ (US); Wayne Arthur Yates, San Mateo, CA (US)

(21) Appl. No.: 18/862,868

(22) PCT Filed: May 4, 2023
(86) PCT No.: PCT/US2023/021004

§ 371 Date: Nov. 4, 2024

Related U.S. Application Data
(60) Provisional application No. 63/338,621, filed on May 5, 2022.

(51) Int. Cl. A61K 31/437; A61K 9/00; A61K 47/02; A61K 47/12

(52) U.S. Cl. CPC A61K 31/437; A61K 9/0078; A61K 47/02; A61K 47/12

(57) ABSTRACT

Provided herein is a method of treating a subject with a respiratory disorder when the subject is receiving high-flow oxygen through a nasal cannula, comprising administering a pharmaceutical composition comprising a therapeutic agent (such as nezulcitinib), or a pharmaceutically acceptable salt thereof, to the subject by oral nebulized inhalation.

### CROSS-REFERENCE TO RELATED APPLICATIONS

This application claims the benefit under 35 U.S.C. § 119(e) of U.S. Provisional Application No. 63/338,621, filed May 5, 2022, which is hereby incorporated by reference in its entirety.

### BACKGROUND

### Field

Provided herein is a method of treating a subject with a respiratory disorder when the subject is receiving high-flow oxygen through a nasal cannula at a rate greater than or equal to about 30 L/min, the method comprising administering a pharmaceutical composition comprising a therapeutic agent (such as a JAK inhibitor), or a pharmaceutically acceptable salt thereof, to the subject by oral nebulized inhalation. Also provided herein is a method of treating a subject with a respiratory disorder when the subject is receiving high-flow oxygen through a nasal cannula at a rate greater than or equal to about 30 L/min, the method comprising administering a pharmaceutical composition comprising nezulcitinib, or a pharmaceutically acceptable salt thereof, to the subject by oral nebulized inhalation.

### State of the Art

For subjects suffering from respiratory disorders, ambient atmospheric oxygen concentration may not be adequate to prevent hypoxemia. Providing supplemental oxygen, wherein a subject receives a supply of oxygen at a higher rate or in a higher concentration than that which is present atmospherically, can help normalize subject blood oxygen saturation. The delivery method for supplemental oxygen depends upon subject need, with low-flow oxygen supplying a lower dose via nasal cannula or simple face mask, usually at a rate of about 4-8 L/min, and high-flow oxygen supplying a more concentrated dose via non-invasive ventilation or high-flow nasal cannula, at a rate of about 30-60 L/min.

However, subjects suffering from various respiratory disorders, including, but not limited to, asthma, chronic obstructive pulmonary disease (COPD), cystic fibrosis (CF), pneumonitis, interstitial lung diseases (including idiopathic pulmonary fibrosis), acute lung injury, acute respiratory distress syndrome, bronchitis, emphysema, sarcoidosis, bronchiolitis obliterans organizing pneumonia (also termed COS), Primary Graft Dysfunction (PGD), acute rejection (AR), lymphocytic bronchiolitis (LB), Chronic Lung Allograft Dysfunction (CLAD), graft-versus-host disease (GVHD), and coronavirus infection, could also benefit from the administration of inhaled pharmaceutical compositions, such as those comprising JAK inhibitors.

The administration of inhaled pharmaceutical compositions concomitantly with the administration of high-flow supplemental oxygen via a nasal cannula can pose a difficulty (and as such, was not used heretofore in human subjects) in adequately delivering the pharmaceutical composition to the respiratory system of the subject while maintaining the increased flow of oxygen into the subject's lungs. Various references, utilizing various models, suggest this difficulty can be addressed by either decreasing the flow of oxygen during administration of the pharmaceutical composition, or by administering the pharmaceutical composition in-line with the flow of oxygen, so that the pharmaceutical composition is delivered through the nasal cannula to the subject. See, for example, Reminiac et al. J. Aerosol Med. Pulm. Drug Deliv. 2016, 29(2):134-141; Alconforado et al., Pharmaceutics, 2019, 11, 320; Li et al, Pharmaceutics, 2019, 11, 225; and Ari et al, Can. J. Respir. Ther., 2021, 57:22-25.

Subjects affected by respiratory disorders would benefit from both therapeutic intervention and supplemental oxygen. Therefore, a need remains for methods for treating a subject having a respiratory disorder with a therapeutic agent, such as a JAK inhibitor, wherein said subject is also receiving high-flow supplemental oxygen via a nasal cannula.

### SUMMARY

The administration of high-flow supplemental oxygen to a subject suffering from a respiratory disorder can create positive pressure in the subject's respiratory system, impeding effective delivery of an inhaled pharmaceutical composition throughout the subject's airways and lungs. Current recommendations for the optimal administration of an inhaled aerosolized pharmaceutical composition to subjects also receiving nasal high-flow supplemental oxygen specify either that (a) the flow rate of supplemental oxygen should be decreased during the administration, to improve inhaled drug delivery, then returned to the high flow rate upon completion of administration, and/or (b) the pharmaceutical composition should be administered in-line through the nasal cannula high-flow supplemental oxygen system. However, it has now been discovered that a pharmaceutical composition of a JAK inhibitor (such as nezulcitinib) can be optimally administered to a subject in need thereof via nebulized oral inhalation, while said subject also receives nasal high-flow supplemental oxygen at an uninterrupted rate greater than or equal to about 30 L/min through a high-flow nasal cannula throughout administration of the pharmaceutical composition.

Provided herein is a method for treating a respiratory disorder in a subject in need thereof when the subject is receiving high-flow oxygen through a nasal cannula, the method comprising administering a pharmaceutical composition to the subject by oral nebulized inhalation while the subject is receiving high-flow oxygen through a nasal cannula at a rate greater than or equal to about 30 L/min. In some embodiments, the pharmaceutical composition comprises a JAK inhibitor (such as nezulcitinib) or a pharmaceutically acceptable salt thereof.

Also provided herein is a method for treating a SARS-CoV-2 lung infection in a subject in need thereof when the subject is receiving high-flow oxygen through a nasal cannula, the method comprising administering a pharmaceutical composition to the subject by oral nebulized inhalation while the subject is receiving high-flow oxygen through a nasal cannula at a rate greater than or equal to about 30 L/min. In some embodiments, the pharmaceutical composition comprises a JAK inhibitor (such as nezulcitinib) or a pharmaceutically acceptable salt thereof.

### BRIEF DESCRIPTION OF THE DRAWINGS

FIG. 1 shows subject plasma concentration of nezulcitinib as a function of time, upon receiving a 3 mg dose via nebulized oral inhalation, while said subject also receives low-flow oxygen at a rate of 6 L/min by nasal cannula.

FIG. 2 shows subject plasma concentration of nezulcitinib as a function of time, comparing route of therapeutic administration and rate of supplemental oxygen supply, namely receiving a 3 mg dose via nebulized oral inhalation while also receiving nasal low-flow oxygen at a rate of 6 L/min, versus receiving a 3 mg dose via nasal inhalation while also receiving high-flow oxygen via nasal cannula circuit at a rate of 30 L/min.

FIG. 3 shows subject plasma concentration of nezulcitinib as a function of time, further comparing route of therapeutic administration and rate of supplemental oxygen supply, specifically receiving a 3 mg dose via nebulized oral inhalation while also receiving nasal low-flow oxygen at a rate of 6 L/min, receiving a 3 mg dose via nasal inhalation while also receiving high-flow oxygen via nasal cannula circuit at a rate of 30 L/min, or receiving a 3 mg dose via nebulized oral inhalation while also receiving high-flow oxygen via nasal cannula at a rate of 30 L/min.

FIG. 4 shows subject plasma concentration of nezulcitinib as a function of time, upon receiving a 3 mg dose via nebulized oral inhalation while also receiving either nasal low-flow oxygen at a rate of 6 L/min or high-flow oxygen via a high-flow nasal cannula at a rate of 30 L/min.

FIG. 5 shows subject plasma concentration of nezulcitinib as a function of time, upon receiving a 3 mg dose via nebulized oral inhalation while also receiving either nasal low-flow oxygen at a rate of 6 L/min, high-flow oxygen via a high-flow nasal cannula at a rate of 30 L/min, or high-flow oxygen via a high-flow nasal cannula at a rate of 50 L/min.

FIG. 6 shows subject plasma concentration of nezulcitinib as a function of time, upon receiving a 3 mg dose via nebulized oral inhalation while also receives either nasal low-flow oxygen at a rate of 6 L/min, high-flow oxygen via a high-flow nasal cannula at a rate of 30 L/min, or high-flow oxygen via a high-flow nasal cannula at a rate of 50 L/min, compared with a subject receiving a 9 mg dose of nezulcitinib via nebulized oral inhalation while also receiving high-flow oxygen via a high-flow nasal cannula at a rate of 50 L/min.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### Definitions

When describing this disclosure, including its various aspects and embodiments, the following terms have the following meanings, unless otherwise indicated.

The term “about” means±5 percent of the specified value.

The term “AUC₀₋₂₄” means the area under the plasma concentration versus time curve, from time zero to 24 hours post-dose.

The term “AUC_{0-∞}” means area under the plasma concentration-time curve from time 0 extrapolated to infinity.

The term “Cₘₐₓ” means maximum observed plasma concentration.

The term “PK” means pharmacokinetic.

The term “Tₘₐₓ” means time to maximum plasma concentration.

The term “t_{1/2}” means apparent terminal elimination half-life.

The term “therapeutically effective amount” means an amount sufficient to effect treatment when administered to a subject in need of treatment.

The term “treating” or “treatment” means ameliorating or suppressing the medical condition, disease or disorder being treated in a subject (particularly a human); or alleviating the symptoms of the medical condition, disease, or disorder.

“Subject” or “patient” refers to an animal, such as a mammal, that has been or will be the object of treatment, observation, or experiment. The methods described herein may be useful in both human therapy and veterinary applications. In some embodiments, the subject or patient is a mammal. In some embodiments, the subject or patient is human.

The term “pharmaceutically acceptable salt” means a salt that is acceptable for administration to a subject or a mammal, such as a human (e.g., salts having acceptable mammalian safety for a given dosage regime). Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

The term “salt thereof” means a compound formed when the hydrogen of an acid is replaced by a cation, such as a metal cation or an organic cation and the like. For example, the cation can be a protonated form of a compound of formula 1, i.e. a form where one or more amino groups have been protonated by an acid. Typically, the salt is a pharmaceutically acceptable salt, although this is not required for salts of intermediate compounds that are not intended for administration to a subject.

Provided herein is a method for treating a lung disease, disorder, or condition in a subject in need thereof when the subject is receiving high-flow oxygen through a nasal cannula, the method comprising administering a pharmaceutical composition comprising a therapeutic agent, or a pharmaceutically acceptable salt thereof, to the subject by oral nebulized inhalation while the subject is receiving high-flow oxygen through a nasal cannula at a rate greater than or equal to about 30 L/min.

It is contemplated that administration of a pharmaceutical composition comprising a therapeutic agent as described herein to a subject by oral nebulized inhalation while the subject is receiving high-flow oxygen through a nasal cannula at a rate greater than or equal to about 30 L/min is unexpectedly superior to administration through the in-line high-flow circuit, contrary to current recommendations in the art. Moreover, it is contemplated that administration by oral nebulized inhalation to subjects receiving high-flow and low-flow oxygen resulted surprisingly in similar exposure.

In some embodiments, the lung disease, disorder, or condition is an inflammatory disease or a fibrotic disease of the respiratory tract. In some embodiments, the lung disease, disorder, or condition is a respiratory disorder. In some embodiments, the lung disease, disorder, or condition is lung transplant rejection. In some embodiments, the lung disease, disorder, or condition is asthma.

In some embodiments, the respiratory disorder is a viral infection, lung transplant rejection, asthma, chronic obstructive pulmonary disease, cystic fibrosis, pneumonitis, idiopathic pulmonary fibrosis, acute lung injury, acute respiratory distress syndrome, bronchitis, emphysema, bronchiolitis obliterans, sarcoidosis, an eosinophilic disease, a helminthic infection, pulmonary arterial hypertension, lymphangioleiomyomatosis, bronchiectasis, an infiltrative pulmonary disease, drug-induced pneumonitis, fungal induced pneumonitis, allergic bronchopulmonary aspergillosis, hypersensitivity pneumonitis, eosinophilic granulomatosis with polyangiitis, idiopathic acute eosinophilic pneumonia, idiopathic chronic eosinophilic pneumonia, hypereosinophilic syndrome, Luffler's syndrome, bronchiolitis obliterans organizing pneumonia, lung graft-versus-host disease, and immune-checkpoint-inhibitor induced pneumonitis.

In some embodiments, the viral infection is influenza. In some embodiments, the viral infection is a coronavirus infection.

In some embodiments, the therapeutic agent is any therapeutic agent described herein, such as the additional therapeutic agents described herein. In some embodiments, the therapeutic agent is a JAK inhibitor. In some embodiments, the therapeutic agent is an inhaled JAK inhibitor. Non-limiting examples of an inhaled JAK inhibitor include GDC-0214/RG6151, GDC-4379/RG6244, TD-8236, AZD0449, AZD4604, and KN-002. In some embodiments, the JAK inhibitor is suitable for local administration to the lungs, such as for treatment of respiratory disease (i.e. is “a lung-selective JAK inhibitor”). Such compounds are known in the art and include, for example, TD-8236 (which is disclosed in U.S. Pat. No. 10,100,049). In some embodiments, the JAK inhibitor is compound 1.

Provided herein is a method for treating a respiratory disorder in a subject in need thereof when the subject is receiving high-flow oxygen through a nasal cannula, the method comprising administering a pharmaceutical composition comprising a JAK inhibitor, or a pharmaceutically acceptable salt thereof, to the subject by oral nebulized inhalation while the subject is receiving high-flow oxygen through a nasal cannula at a rate greater than or equal to about 30 L/min. In some embodiments, the JAK inhibitor is a lung-selective JAK inhibitor. In some embodiments, the JAK inhibitor is compound 1.

Compound 1 (also referred to as compound of formula 1 and known as nezulcitinib, ((S)-(3-(dimethylamino)azetidin-1-yl)(2-(6-(2-ethyl-4-hydroxyphenyl)-1H-indazol-3-yl)-5-isopropyl-4,5,6,7-tetrahydro-3H-imidazo[4,5-c]pyridin-6-yl)methanone)), having the structure:

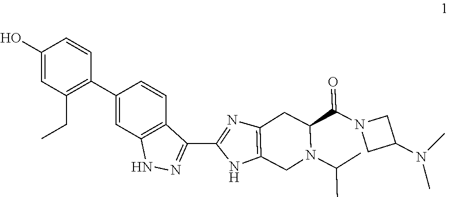 [SMILES: CCc1cc(O)ccc1-c1ccc2c(-c3nc4c([nH]3)CN(C(C)C)[C@H](C(=O)N3CC(N(C)C)C3)C4)n[nH]c2c1]

is a pan-JAK inhibitor suitable for direct delivery to the lungs, which is disclosed in U.S. Pat. No. 10,947,229. Nezulcitinib may be prepared, for example, as described in U.S. Pat. No. 10,947,229, and in U.S. Pat. No. 10,844,057.

Provided herein is a method for treating a respiratory disorder in a subject in need thereof when the subject is receiving high-flow oxygen through a nasal cannula, the method comprising administering a pharmaceutical composition comprising nezulcitinib, or a pharmaceutically acceptable salt thereof, to the subject by oral nebulized inhalation while the subject is receiving high-flow oxygen through a nasal cannula at a rate greater than or equal to about 30 L/min.

In some embodiments, the respiratory disorder is selected from the group consisting of a coronavirus infection, lung transplant rejection, asthma, chronic obstructive pulmonary disease, cystic fibrosis, pneumonitis, idiopathic pulmonary fibrosis, acute lung injury, acute respiratory distress syndrome, bronchitis, emphysema, bronchiolitis obliterans, sarcoidosis, an eosinophilic disease, a helminthic infection, pulmonary arterial hypertension, lymphangioleiomyomatosis, bronchiectasis, an infiltrative pulmonary disease, drug-induced pneumonitis, fungal induced pneumonitis, allergic bronchopulmonary aspergillosis, hypersensitivity pneumonitis, eosinophilic granulomatosis with polyangiitis, idiopathic acute eosinophilic pneumonia, idiopathic chronic eosinophilic pneumonia, hypereosinophilic syndrome, Luffler's syndrome, bronchiolitis obliterans organizing pneumonia, lung graft-versus-host disease, and immune-checkpoint-inhibitor induced pneumonitis. In some embodiments, the respiratory disorder is asthma or chronic obstructive pulmonary disease. In some embodiments, the respiratory disorder is asthma. In some embodiments, the respiratory disorder is chronic obstructive pulmonary disease. In some embodiments, the respiratory disorder is lung transplant rejection. In some embodiments, the respiratory disorder is a coronavirus infection.

Also provided herein is a method for treating a SARS-CoV-2 lung infection in a subject in need thereof when the subject is receiving high-flow oxygen through a nasal cannula, the method comprising administering a pharmaceutical composition comprising nezulcitinib, or a pharmaceutically acceptable salt thereof, to the subject by oral nebulized inhalation while the subject is receiving high-flow oxygen through a nasal cannula at a rate greater than or equal to about 30 L/min.

In some embodiments, the coronavirus infection is caused by SARS-CoV-1, SARS-CoV-2, or MERS-CoV. In some embodiments, the coronavirus infection is caused by SARS-CoV-1. In some embodiments, the coronavirus infection is caused by SARS-CoV-2. In some embodiments, the coronavirus infection is caused by MERS-CoV.

In some embodiments, the subject has acute lung injury associated with COVID-19. In some embodiments, the subject does not have acute lung injury associated with COVID-19.

In some embodiments, the subject has a baseline level of C-reactive protein less than or equal to about 150 mg/L.

In some embodiments, the method reduces coronavirus viral load in the respiratory system of the subject. In some embodiments, the method reduces coronavirus viral load in the lungs of the subject. In some embodiments, the reduction in coronavirus viral load is measured by collecting and analyzing nasal swabs from the subject. In some embodiments, the method reduces the viral load of SARS-CoV-2 in the respiratory system of the subject. In some embodiments, the method reduces the viral load of SARS-CoV-2 in the lungs of the subject. In some embodiments, the reduction in viral load of SARS-CoV-2 is measured by collecting and analyzing nasal swabs from the subject.

In some embodiments, the subject suffers from one or more of hypoxia, hypoxemia, dyspnea, shortness of breath, and low oxygen levels.

In some embodiments, the pharmaceutical composition is administered to the subject in an outpatient setting. In some embodiments, the pharmaceutical composition is administered to the subject, wherein the subject is not hospitalized. In some embodiments, the pharmaceutical composition is administered to the subject before hospitalization. In some embodiments, the pharmaceutical composition is administered to the subject during hospitalization.

In some embodiments, the method results in a decreased time to recovery and/or time to discharge from a hospital or medical facility. In some embodiments, the method results in a decrease in hospitalization time, a decrease in time in the 1CU, and/or a decrease in time to discharge.

In some embodiments, the subject is receiving high-flow oxygen through a nasal cannula at a rate of about 30 L/min to about 50 mL/min. In some embodiments, the subject is receiving high-flow oxygen through a nasal cannula at a rate of about 30 L/min. In some embodiments, the subject is receiving high-flow oxygen through a nasal cannula at a rate of about 50 mL/min.

In some embodiments, the pharmaceutical composition comprises about 1 mg to about 10 mg of nezulcitinib, or a pharmaceutically acceptable salt thereof. In some embodiments, the pharmaceutical composition comprises about 3 mg to about 9 mg of nezulcitinib, or a pharmaceutically acceptable salt thereof. In some embodiments, the pharmaceutical composition comprises about 3 mg of nezulcitinib, or a pharmaceutically acceptable salt thereof. In some embodiments, the pharmaceutical composition comprises about 9 mg of nezulcitinib, or a pharmaceutically acceptable salt thereof.

In some embodiments, the pharmaceutical composition comprises about 1 mg/l mL to about 10 mg/l mL (free base equivalent) of nezulcitinib. In some embodiments, the pharmaceutical composition comprises about 3 mg/l mL to about 9 mg/l mL (free base equivalent) of nezulcitinib. In some embodiments, the pharmaceutical composition comprises about 3 mg/l mL (free base equivalent) of nezulcitinib. In some embodiments, the pharmaceutical composition comprises about 3 mg/l mL (free base equivalent) of nezulcitinib or a pharmaceutically acceptable salt thereof. In some embodiments, the pharmaceutical composition comprises about 9 mg/l mL (free base equivalent) of nezulcitinib.

In some embodiments, the pharmaceutical composition has a pH in the range of about 3.5 to about 4.5. In some embodiments, the pharmaceutical composition has a pH of about 4.0.

In some embodiments, the pharmaceutical composition further comprises a buffering agent. In some embodiments, the buffering agent is citric acid.

In some embodiments, the pharmaceutical composition further comprises a tonicity agent. In some embodiments, the tonicity agent is sodium chloride.

In some embodiments, the pharmaceutical composition is administered by inhalation. In some embodiments, the pharmaceutical composition is administered by nebulized inhalation. In some embodiments, the pharmaceutical composition is administered by oral nebulized inhalation.

In some embodiments, the pharmaceutical composition is administered once a day. In some embodiments, the pharmaceutical composition is administered twice a day.

In some embodiments, the pharmaceutical composition is administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 28 days, 2 weeks, 3 weeks, 4 weeks, 1 month, 5 weeks, 6 weeks, 2 months, or 3 months. In some embodiments, the pharmaceutical composition is administered until discharge of the subject from the hospital. In some embodiments, the pharmaceutical composition is administered to the subject for up to 7 days, or up to 10 days, or up to 14 days, or up to 21 days, or up to 28 days, or until discharge from the hospital. In some embodiments, the pharmaceutical composition is administered to the subject for up to 7 days, or until discharge from the hospital. In some embodiments, the pharmaceutical composition is administered to the subject for up to 14 days, or until discharge from the hospital. In some embodiments, the pharmaceutical composition is administered to the subject for up to 21 days, or until discharge from the hospital. In some embodiments, the pharmaceutical composition is administered to the subject for up to 28 days, or until discharge from the hospital.

In some embodiments, the pharmaceutical composition is administered at a higher loading dose on day 1 of administration, followed by a lower dose on the following days.

In some embodiments, the method comprises administering one or more additional therapeutic agents or treatments to the subject. In some embodiments, the additional therapeutic agent is an antiviral agent, an anti-inflammatory agent, or an antibody. In some embodiments, the additional therapeutic agent is an antiviral agent. In some embodiments, the additional therapeutic agent is an anti-inflammatory agent. In some embodiments, the additional therapeutic agent is an antibody.

In some embodiments, the method results in an increase in oxygen levels in the blood of the subject. In some embodiments, the method results in an improvement or resolution of fever in the subject. In some embodiments, the method results in removal of the subject from oxygen supplementation, or prevents the need for the subject to be placed on a ventilator. In some embodiments, the method increases the number of ventilator-free days (VFDs) in the subject. In some embodiments, the method increases ICU (Intensive Care Unit) free days for the subject. In some embodiments, the method results in an improvement or resolution of shortness of breath. In some embodiments, the method results in a lower risk of mortality in the subject.

In some embodiments, the method decreases inflammation in the lungs caused by the respiratory disorder. In some embodiments, the method prevents, reduces, or resolves acute lung injury and/or acute respiratory distress syndrome caused by the respiratory disorder.

In some embodiments, the method decreases inflammation in the lungs caused by a coronavirus infection. In some embodiments, the method prevents, reduces, or resolves acute lung injury and/or acute respiratory distress syndrome caused by a coronavirus infection. In some embodiments, the method prevents, reduces, or stops a cytokine storm caused by a coronavirus infection. In some embodiments, the method results in an increase in oxygen levels in the blood of the subject. In some embodiments, the method results in an improvement or resolution of fever in the subject.

In some embodiments, the method decreases inflammation in the lungs caused by COVID-19. In some embodiments, the method prevents, reduces, or resolves acute lung injury and/or acute respiratory distress syndrome caused by COVID-19. In some embodiments, the method prevents, reduces, or stops a cytokine storm caused by COVID-19. In some embodiments, the method results in an increase in oxygen levels in the blood of the subject. In some embodiments, the method results in an improvement or resolution of fever in the subject. In some embodiments, the method results in removal of the subject from oxygen supplementation, or prevents the need for the subject to be placed on a ventilator. In some embodiments, the method increases the number of ventilator-free days in the subject. In some embodiments, the method increases ICU (Intensive Care Unit) free days for the subject. In some embodiments, the method results in an improvement or resolution of shortness of breath. In some embodiments, the method results in a lower risk of mortality in the subject.

In some embodiments, the method prevents hospitalization of the subject. In some embodiments, the method prevents serious complications in the subject. In some embodiments, the serious complications include, for example, lung injury, acute lung injury (ALI), acute respiratory distress syndrome (ARDS), organ failure, pneumonia, acute liver injury, blood clots, respiratory failure, further need for oxygen supplementation, non-invasive ventilation, mechanical ventilation, acute cardiac injury, a secondary infection, acute kidney injury, septic shock, disseminated intravascular coagulation, Multisystem Inflammatory Syndrome (MIS-C), rhabdomyolysis, arrhythmia, a cytokine storm, and cardiovascular shock.

In some embodiments, the subject is at a high risk of developing severe complications from the coronavirus. In some embodiments, the subject has been identified as being at a high risk of developing severe complications from the coronavirus through biomarker testing. In some embodiments, the subject has been identified as being at a high risk of developing severe complications from the coronavirus based on LDH (lactate dehydrogenase) levels. In some embodiments, the subject has been identified as being at a high risk of developing severe complications from the coronavirus based on LDH-isoform3 levels. In some embodiments, the subject has been identified as being at a high risk of developing severe complications from the coronavirus based on levels of Surfactant Protein-D (SPD), Receptor for Advanced Glycation End-products (RAGE), one or more cytokines, C-reactive protein (CRP), D-dimer, fibrinogen, and/or ferritin. In some embodiments, the cytokine is IL-6. In some embodiments, the subject has diabetes, obesity, a cardiovascular disease (such as a coronary artery disease, myocardial infarction, a history of cerebrovascular accident, or peripheral arterial disease), hypertension, or a lung disease. In some embodiments, the subject has coronary artery disease, myocardial infarction, a history of cerebrovascular accident, peripheral arterial disease, a lung disease (such as asthma, COPD, or idiopathic pulmonary fibrosis (IPF)). In some embodiments, the subject has been identified as being at a high risk of developing severe complications from the coronavirus based on a chest x-ray. In some embodiments, the subject has a CXR abnormality consistent with viral pneumonia.

In some embodiments, the method decreases the rate of medical intervention associated with the coronavirus infection. In some embodiments, the rate of medical intervention associated with the coronavirus infection is measured by the number of emergency visits, hospitalizations, physician visits, and urgent care visits associated with the coronavirus infection.

In some embodiments, the pharmaceutical composition inhibits viral entry or fusion of the coronavirus virions with the endosomal membrane in the cells of the subject. In some embodiments, the pharmaceutical composition inhibits an Abelson kinase in the subject. In some embodiments, the pharmaceutical composition inhibits Abelson kinases in the subject. In some embodiments, the Abelson kinases are Abl1 and Abl2. In some embodiments, the Abelson kinase is Abl1. In some embodiments, the Abelson kinase is Abl2. In some embodiments, the pharmaceutical composition inhibits replication of the coronavirus in the subject.

In some embodiments, the subject has COVID-19-associated inflammatory syndrome (i.e. Multisystem Inflammatory Syndrome, MIS-C). In some embodiments, the subject is a pediatric subject.

In some embodiments, the method prevents long-term lung dysfunction or “long haul COVID” in the subject.

In some embodiments, the method results in an increase in the number of RFDs (respiratory failure-free days) for a subject infected with a coronavirus. In some embodiments, the method results in an increase in the number of ventilator-free days (VFDs) for a subject infected with coronavirus.

In some embodiments, the method increases the ratio of arterial oxygen partial pressure to fractional inspired oxygen (PaO₂/FiO₂) in the subject.

In some embodiments, the method decreases the mortality rate in the subject population.

In some embodiments, the method decreases blood clot formation in the subject.

In some embodiments, the method results in a decrease for the need for oxygen supplementation, non-invasive ventilation, or mechanical ventilation in the subject.

In some embodiments, the subject is classified as moderate, severe, or critical. In some embodiments, the subject is 60 years old or less. In some embodiments, the subject is older than 60 years old. In some embodiments, the subject suffers from pneumonia when the pharmaceutical composition is administered. In some embodiments, the subject suffers from bilateral pneumonia when the pharmaceutical composition is administered. In some embodiments, the administration of the pharmaceutical composition results in: prevention or attenuation of the formation of lung lesions/lung lesion opacity/lung injury, improvement in the subject monitored by chest imaging, CT scan, or chest x-ray, increase in the number of ventilator-free days (VFDs), increase in the number of ICU-free days, increase in PaO₂/FiO₂ ratio, or increase in SaO₂/FiO₂ ratio. In some embodiments, administration of the pharmaceutical composition to a subject infected with coronavirus is done when the subject is in respiratory failure, but before mechanical ventilation is needed.

In some embodiments, the method results in an increase in days without supplemental oxygen for the subject.

In some embodiments, the pharmaceutical composition is administered to the subject at a key inflection point of the disease, before ALI develops, and prevents progression to ALI. In some embodiments, the pharmaceutical composition is administered to the subject during the early stages of the coronavirus infection, before ALI develops, and the administration prevents progression to ALI. In some embodiments, the pharmaceutical composition is administered for a short period of time. In some embodiments, the pharmaceutical composition is administered to the subject before ARDS develops, and prevents progression to ARDS. In some embodiments, administration of the pharmaceutical composition to a subject infected with coronavirus is done before the subject has ALI and/or ARDS, in order to prevent ALI and/or ARDS in the subject.

In some embodiments, the subject has one or more underlying conditions, such as asthma, COPD, a cardio-vascular disease, diabetes, chronic lung disease, a heart condition, cancer, a bone marrow or organ transplantation, an immune deficiency, HIV, takes an immune weakening medication, obesity, chronic kidney disease, a neurodevelopmental condition, high blood pressure, or liver disease.

In some embodiments, the subject is 80 years old or older, 70 years old or older, 65 years old or older, 60 years old or older, 50 years old or older, 40 years old or older, 10 years old or younger, between and 20 years old, between 20 and 30 years old, between 30 and 40 years old, between 40 and 50 years old, between 50 and 60 years old, between 20 and 40 years old, between 40 and 60 years old, between 60 and 80 years old, 60 years old or younger, or over 60 years old. In some embodiments, the subject is 16 years old or older. In some embodiments, the subject is 18 years old or older. In some embodiments, the subject is 12 years old or older.

In some embodiments, the method blocks or inhibits neutrophilia and/or the formation of neutrophil extracellular traps (NETs) in the subject.

In some embodiments, the method decreases the risk of thrombosis or thromboembolism in the subject.

In some embodiments, the method decreases the incidence of thrombosis or thromboembolism in the subject population.

In some embodiments, the maximum plasma concentration (Cmax) in the subject of the compound of formula 1 is under 350 ng/mL. In some embodiments, the maximum plasma concentration in the subject of the compound of formula 1 is under 300 ng/mL. In some embodiments, the maximum plasma concentration in the subject of the compound of formula 1 is under 250 ng/mL. In some embodiments, the maximum plasma concentration in the subject of the compound of formula 1 is under 200 ng/mL. In some embodiments, the maximum plasma concentration in the subject of the compound of formula 1 is under 150 ng/mL. In some embodiments, the maximum plasma concentration in the subject of the compound of formula 1 is under 100 ng/mL. In some embodiments, the maximum plasma concentration in the subject of the compound of formula 1 is under 50 ng/mL. In some embodiments, the maximum plasma concentration in the subject of the compound of formula 1 is under 40, 30, 25, 20, 15, or 10 ng/mL. In some embodiments, the maximum plasma concentration in the subject of the compound of formula 1 is under the plasma concentration necessary to achieve JAK IC₅₀. In some embodiments, the JAK IC₅₀ is calculated by determining the IC₅₀ for IL-13-induced STAT6 phosphorylation in the human bronchial epithelial cell line BEAS-2B. In some embodiments, the maximum plasma concentration in the subject of the compound of formula 1 is under the plasma concentration necessary to inhibit Janus kinases by 50%. In some embodiments, the compound of formula 1, or a pharmaceutically acceptable salt thereof, is administered to the subject at a dose of about 1 mg to about 10 mg. In some embodiments, the compound of formula 1, or a pharmaceutically acceptable salt thereof, is administered to the subject at a dose of about 1 mg. In some embodiments, the compound of formula 1, or a pharmaceutically acceptable salt thereof, is administered to the subject at a dose of about 3 mg. In some embodiments, the compound of formula 1, or a pharmaceutically acceptable salt thereof, is administered to the subject at a dose of about 9 mg. In some embodiments, the compound of formula 1, or a pharmaceutically acceptable salt thereof, is administered to the subject at a dose of about 10 mg. In some embodiments, the compound of formula 1, or a pharmaceutically acceptable salt thereof, is administered to the subject at a dose of about 1 mg to about 3 mg. In some embodiments, the compound of formula 1, or a pharmaceutically acceptable salt thereof, is administered to the subject at a dose of about 3 mg to about 10 mg. In some embodiments, the compound of formula 1, or a pharmaceutically acceptable salt thereof, is administered to the subject at a dose of about 2 mg, or about 4 mg, or about 5 mg, or about 6 mg, or about 7 mg, or about 8 mg, or about 9 mg. In some embodiments, the compound of formula 1, or a pharmaceutically acceptable salt thereof, is administered once a day. In some embodiments, the compound of formula 1, or a pharmaceutically acceptable salt thereof, is administered twice a day. In some embodiments, the compound of formula 1, or a pharmaceutically acceptable salt thereof, is administered at twice the dose on the first day.

In some embodiments, the administration of the compound of formula 1, or a pharmaceutically acceptable salt thereof, results in a plasma AUC₀₋₂₄ under 500 ng*hr/mL, or under 250 ng*hr/mL, or under 100 ng*hr/mL, or under 50 ng*hr/mL.

In some embodiments, the administration of the compound of formula 1, or a pharmaceutically acceptable salt thereof, results in a Tₘₐₓ between 0.5 hr and 4 hr, or between 0.5 and 2 hr, or a Tₘₐₓ of about 1 hr.

In some embodiments, the subject is symptomatic. In some embodiments, the subject is hospitalized. In some embodiments, the subject requires supplemental oxygen. In some embodiments, the subject has acute lung injury associated with COVID-19.

In some embodiments, the subject is 12 years old or older. In some embodiments, the subject is under 12 years old. In some embodiments, the subject is a pediatric subject, two years of age or older.

In some embodiments, the subject has mild to moderate COVID-19. In some embodiments, the subject has severe COVID-19. In some embodiments, the subject is at high risk for progressing to severe COVID-19 and/or hospitalization.

In some embodiments, the method results in an improvement in the Patient Global Assessment of Symptoms of the subject. In some embodiments, the method results in an improvement in the Patient Global Rating of Change of the subject. In some embodiments, the method results in an improvement in levels of LDH (lactate dehydrogenase), Surfactant Protein-D (SPD), Receptor for Advanced Glycation End-products (RAGE), one or more cytokines, C-reactive protein (CRP), D-dimer, fibrinogen, and/or ferritin in the subject. In some embodiments, the method improves the Borg Dyspnea Score in the subject. In some embodiments, the method decreases C-Reactive protein levels (CRP) in the subject. In some embodiments, the method decreases D-dimer levels in the subject. In some embodiments, the method decreases cytokine levels in the subject. In some embodiments, the cytokine is IL-6.

In some embodiments, the method results in an improvement in the levels of Receptor for Advanced Glycation End-products (RAGE) in the subject. In some embodiments, the method results in a decrease in the levels of Receptor for Advanced Glycation End-products (RAGE) in the subject. In some embodiments, the method results in a decrease in lung injury to the subject.

In some embodiments, the method results in an improvement in the levels of C-reactive protein (CRP) in the subject. In some embodiments, the method results in a decrease in the levels of C-reactive protein (CRP) in the subject. In some embodiments, the method results in an improvement in the levels of IL-6 in the subject. In some embodiments, the method results in a decrease in the levels of IL-6 in the subject. In some embodiments, the method results in an improvement in the levels of IFNγ in the subject.

In some embodiments, the method results in a decrease in the levels of IFNγ in the subject. In some embodiments, the method results in an improvement in the levels of IP-10 in the subject. In some embodiments, the method results in a decrease in the levels of IP-10 in the subject. In some embodiments, the method results in a decrease in the levels of IL-10 in the subject. In some embodiments, the method results in a decrease in the levels of MCP-1 in the subject. In some embodiments, the method results in an improvement in the modified Borg Dyspnea Score for the subject.

In some embodiments, the method results in a decrease in the mortality risk for the subject.

In some embodiments, the method comprises administering one or more additional therapeutic agents or treatments to the subject. In some embodiments, the subject receives standard of care co-treatment. In some embodiments, the subject is also treated with corticosteroids. In some embodiments, the subject is also treated with dexamethasone. In some embodiments, the subject is also treated with an antiviral agent. In some embodiments, the subject is also treated with antiviral agents. In some embodiments, the subject is also treated with remdesivir.

In some embodiments, the subject suffers from hypertension and/or diabetes.

In some embodiments, the subject suffers from moderate COVID-19 when treatment with compound 1, or a pharmaceutically acceptable salt thereof, is initiated. In some embodiments, the subject suffers from severe COVID-19 when treatment with compound 1, or a pharmaceutically acceptable salt thereof, is initiated.

In some embodiments, the subject has COVID-19 associated acute lung injury. In some embodiments, the subject requires supplemental oxygen when admitted. In some embodiments, the subject requires supplemental oxygen but is not on ventilation or high-flow oxygen when admitted. In some embodiments, the subject requires invasive mechanical ventilation or extracorporeal membrane oxygenation when admitted. In some embodiments, the subject is on non-invasive ventilation or high-flow oxygen devices when admitted.

In some embodiments, the method prevents, reduces, or resolves acute lung injury and/or acute respiratory distress syndrome caused by COVID-19. In some embodiments, the method prevents, reduces, or stops a cytokine storm caused by COVID-19. In some embodiments, the method results in removal of the subject from ventilation or oxygen supplementation.

### Nezulcitinib

Chemical structures are named herein according to IUPAC conventions as implemented in ChemDraw software (PerkinElmer, Inc., Cambridge, MA). Compound 1, nezulcitinib, is designated as (S)-(3-(dimethylamino)azetidin-1-yl)(2-(6-(2-ethyl-4-hydroxyphenyl)-1H-indazol-3-yl)-5-isopropyl-4,5,6,7-tetrahydro-3H-imidazo[4,5-c]pyridin-6-yl)methanone.

Furthermore, the imidazo portion of the tetrahydroimidazopyridine moiety exists in tautomeric forms, illustrated below for a fragment of compound 1

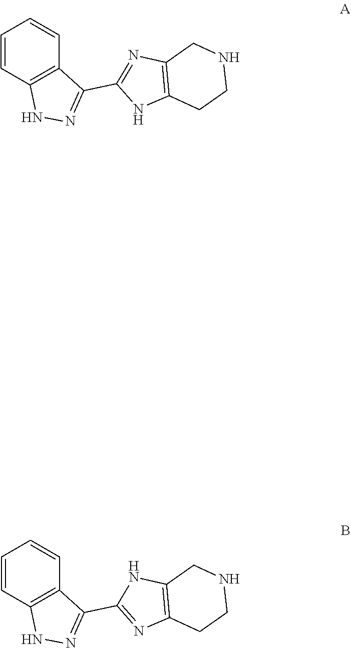 [SMILES: c1ccc2c(-c3nc4c([nH]3)CCNC4)n[nH]c2c1 ; c1ccc2c(-c3nc4c([nH]3)CNCC4)n[nH]c2c1]

According to the IUPAC convention, these representations give rise to different numbering of the atoms of the imidazole portion: (1H-indazol-3-yl)-4,5,6,7-tetrahydro-1H-imidazo[4,5-c]pyridine (structure A) vs. (1H-indazol-3-yl)-4,5,6,7-tetrahydro-3H-imidazo[4,5-c]pyridine (structure B). It will be understood that although structures are shown, or named, in a particular form, the disclosed compounds also include the tautomers thereof.

Compound 1 may exist as a pure enantiomer or as an enriched mixture. The depiction or naming of a particular stereoisomer means the indicated stereocenter has the designated stereochemistry with the understanding that minor amounts of other stereoisomers may also be present unless otherwise indicated, provided that the utility of the depicted or named compound is not eliminated by the presence of another stereoisomer.

Compound 1 also contains several basic groups (e.g., amino groups) and therefore, such compound can exist as the free base or in various salt forms, such a mono-protonated salt form, a di-protonated salt form, a tri-protonated salt form, or mixtures thereof. All such forms are included within the scope of the disclosure, unless otherwise indicated.

This disclosure also includes isotopically-labeled compounds of formula 1, i.e., compounds of formula 1 where one or more atoms have been replaced or enriched with an atom having the same atomic number but an atomic mass different from the atomic mass that predominates in nature. Examples of isotopes that may be incorporated into a compound of formula 1 include, but are not limited to, ²H, ³H, ¹¹C, ¹³C, ¹⁴C, ¹³N, ¹⁵N, 150, 170, and 180.

### Pharmaceutical Compositions

Compound 1 and pharmaceutically acceptable salts thereof are typically used in the form of a pharmaceutical composition or formulation. Such pharmaceutical compositions may advantageously be administered to a subject by inhalation. In addition, pharmaceutical compositions may be administered by any acceptable route of administration including, but not limited to, oral, rectal, nasal, topical (including transdermal) and parenteral modes of administration.

Provided herein are pharmaceutical composition comprising a pharmaceutically acceptable carrier or excipient, and compound 1, where, as defined above, “compound 1” means compound 1, or a pharmaceutically acceptable salt thereof. Optionally, such pharmaceutical compositions may contain other therapeutic and/or formulating agents if desired. When discussing compositions and uses thereof, compound 1 may also be referred to herein as the “active agent.”

The pharmaceutical compositions of the disclosure typically contain a therapeutically effective amount of compound 1. Those skilled in the art will recognize, however, that a pharmaceutical composition may contain more than a therapeutically effective amount, i.e., bulk compositions, or less than a therapeutically effective amount, i.e., individual unit doses designed for multiple administrations to achieve a therapeutically effective amount, or an amount sufficient to effect a desired biological effect, such as decreasing the viral load of a coronavirus.

Typically, such pharmaceutical compositions will contain from about 0.01 to about 95% by weight of the active agent; including, for example, from about 0.05 to about 30% by weight; and from about 0.1% to about 10% by weight of the active agent.

Any conventional carrier or excipient may be used in the pharmaceutical compositions comprising compound 1. The choice of a particular carrier or excipient, or combinations of carriers or excipients, will depend on the mode of administration being used to treat a particular subject or type of medical condition or disease state. In this regard, the preparation of a suitable pharmaceutical composition for a particular mode of administration is well within the scope of those skilled in the pharmaceutical arts. Additionally, the carriers or excipients used in the pharmaceutical compositions of this disclosure are commercially available. By way of further illustration, conventional formulation techniques are described in Remington: The Science and Practice of Pharmacy, 20th Edition, Lippincott Williams & White, Baltimore, Maryland (2000); and H. C. Ansel et al., Pharmaceutical Dosage Forms and Drug Delivery Systems, 7th Edition, Lippincott Williams & White, Baltimore, Maryland (1999).

Representative examples of materials which can serve as pharmaceutically acceptable carriers include, but are not limited to, the following: sugars, such as lactose, glucose, and sucrose; starches, such as corn starch and potato starch; cellulose, such as microcrystalline cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical compositions.

Pharmaceutical compositions are typically prepared by thoroughly and intimately mixing or blending the active agent with a pharmaceutically acceptable carrier and one or more optional ingredients.

In one aspect, the pharmaceutical composition is suitable for inhaled administration. Pharmaceutical compositions for inhaled administration are typically in the form of an aerosol or a powder. Such compositions are generally administered using inhaler delivery devices, such as a dry powder inhaler (DPI), a metered-dose inhaler (MDI), a nebulizer inhaler, or a similar delivery device.

In some embodiments, the pharmaceutical composition is administered by inhalation using a nebulizer inhaler. Such nebulizer devices typically produce a stream of high velocity air that causes the pharmaceutical composition to spray as a mist that is carried into the subject's respiratory tract. Accordingly, when formulated for use in a nebulizer inhaler, the therapeutic agent can be dissolved in a suitable carrier to form a solution. Alternatively, the therapeutic agent can be micronized or nanomilled and combined with a suitable carrier to form a suspension.

A representative pharmaceutical composition for use in a nebulizer inhaler comprises a solution or suspension comprising from about 0.05 g/l mL to about 20 mg/l mL of compound 1 and excipients compatible with nebulized formulations. In some embodiments, the pharmaceutical composition for use in a nebulizer inhaler comprises a solution or suspension comprising from about 1 mg/l mL to about 10 mg/l mL (free base equivalent) of compound 1 and excipients compatible with nebulized formulations. In some embodiments, the pharmaceutical composition for use in a nebulizer inhaler comprises a solution or suspension comprising from about 3 mg/l mL to about 9 mg/l mL (free base equivalent) of compound 1 and excipients compatible with nebulized formulations. In some embodiments, the pharmaceutical composition for use in a nebulizer inhaler comprises a solution or suspension comprising from about 3 mg/l mL (free base equivalent) of compound 1 and excipients compatible with nebulized formulations. In some embodiments, the pharmaceutical composition for use in a nebulizer inhaler comprises a solution or suspension comprising from about 9 mg/l mL (free base equivalent) of compound 1 and excipients compatible with nebulized formulations.

Nebulizer devices suitable for administering therapeutic agents by inhalation are described in the art and examples of such devices are commercially available. For example, representative nebulizer devices or products include the Respimat Softmist Inhaler (Boehringer Ingelheim); the AERx Pulmonary Delivery System (Aradigm Corp.); the PARI LC Plus Reusable Nebulizer (Pari GmbH); and the like.

In some embodiments, the nebulizer is a jet nebulizer, an ultrasonic nebulizer, or a mesh nebulizer. In some embodiments, the nebulizer is a jet nebulizer. In some embodiments, the nebulizer is an ultrasonic nebulizer. In some embodiments, the nebulizer is a mesh nebulizer. In some embodiments, the nebulizer is an active mesh nebulizer. Non-limiting examples of an active mesh nebulizer include Aeroneb (Aerogen) and eFlow (PARI). In some embodiments, the nebulizer is a passive mesh nebulizer. A non-limiting example of a passive mesh nebulizer is Microair NE-US22 (Omron).

The droplet size distribution of a nebulizer is a parameter that can influence the in vivo deposition of the therapeutic agent in the lung. The droplet size can be influenced by the formulation, the nebulizer device, or both. In some embodiments, the delivered atomized particles (i.e. particles of a therapeutic agent delivered via a nebulizer) are comprised of particles substantially having a mean diameter of, for example, from about 1 μm to about 30 μm, about 5 μm to about 30 μm, about 10 μm to about 30 μm, about 15 μm to about 30 μm, about 10 μm to about 20 μm, about 10 μm to about 15 μm, about 1 μm to about 20 μm, about 1 μm to about 10 μm, about 1 μm to about 5 μm, less than about 30 μm, less than about 25 μm, less than about 20 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, or less than about 1 μm.

In some embodiments, the delivered atomized particles are comprised of particles substantially having a mean diameter of about 1 μm to about 10 μm. In some embodiments, the delivered atomized particles are comprised of particles substantially having a mean diameter of about 1 μm to about 10 μm. In some embodiments, the delivered atomized particles are comprised of particles substantially having a mean diameter of less than about 10 μm. In some embodiments, the delivered atomized particles are comprised of particles substantially having a mean diameter of less than about 5 μm.

In some embodiments, about 60% to about 95% of the delivered atomized particles are comprised of particles having a mean diameter of, for example, from about 1 μm to about 5 μm, about 1 μm to about 10 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, or less than about 1 μm. In some embodiments, about 65% to about 95% of the delivered atomized particles are comprised of particles having a mean diameter of, for example, from about 1 μm to about 10 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, or less than about 1 μm. In some embodiments, about 70% to about 90% of the delivered atomized particles are comprised of particles having a mean diameter of, for example, from about 1 μm to about 10 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, or less than about 1 μm. In some embodiments, about 70% to about 85% of the delivered atomized particles are comprised of particles having a mean diameter of, for example, from about 1 μm to about 10 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, or less than about 1 μm. In some embodiments, about 70% to about 80% of the delivered atomized particles are comprised of particles having a mean diameter of, for example, from about 1 μm to about 10 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, or less than about 1 μm.

In some embodiments, administration via nebulizer is powered by an oxygen flow rate, for example, of about 10 L/min to about 1 L/min, about 10 L/min to about 2 L/min, about 8 L/min to about 3 L/min, about 8 less than about 4 L/min, about 8 L/min to about 5 L/min, about 8 L/min to about 6 L/min, less than about 9 L/min, less than about 8 L/min, less than about 7 L/min, less than about 6 L/min, less than about 5 L/min. In some embodiments, the nebulizer is powered by an oxygen flow rate of about 8 L/min to about 6 L/min. In some embodiments, the pharmaceutical composition for use in a nebulizer inhaler has a pH in the range of about 3 to about 8. In some embodiments, the pharmaceutical composition for use in a nebulizer inhaler has a pH in the range of about 3.5 to about 4.5. In some embodiments, the pharmaceutical composition for use in a nebulizer inhaler has a pH of about 4.0.

In some embodiments, the pharmaceutical composition for use in a nebulizer inhaler further comprises a buffering agent. In some embodiments, the buffering agent is citric acid.

In some embodiments, the pharmaceutical composition for use in a nebulizer inhaler further comprises a tonicity agent. In some embodiments, the tonicity agent is sodium chloride.

Provided herein is a method for treating a respiratory disorder in a subject receiving high-flow supplemental oxygen via a nasal cannula in need thereof, comprising administering a pharmaceutical composition comprising nezulcitinib, or a pharmaceutically acceptable salt thereof, to the subject by oral nebulized inhalation, wherein the flow of supplemental oxygen to the subject is not decreased or suspended throughout administration of the pharmaceutical composition.

The following non-limiting examples illustrate representative pharmaceutical compositions of the present disclosure.

### Nebulizer Composition

Compound 1 (25 mg) is dissolved in a solution containing 1.5-2.5 equivalents of hydrochloric acid, followed by addition of sodium hydroxide to adjust the pH to 3.5 to 5.5, and 3% by weight of glycerol. The solution is stirred well until all the components are dissolved. The solution is administered using a nebulizer device that provides about 0.1 mg to about 4 mg of compound 1 per dose.

Compound 1, or a pharmaceutically acceptable salt thereof, will typically be administered in a single daily dose or in multiple doses per day, although other forms of administration may be used. The amount of active agent administered per dose or the total amount administered per day will typically be determined by a physician, in the light of the relevant circumstances, including the condition to be treated, the chosen route of administration, the actual compound administered and its relative activity, the age, weight, and response of the individual subject, the severity of the subject's symptoms, and the like.

### Utility

In some embodiments for the methods provided herein, the therapeutic agent is a JAK inhibitor. In some embodiments, the JAK inhibitor has been designed for the treatment of inflammatory and fibrotic disease of the respiratory tract. In some embodiments, the compounds have been designed to enable delivery of a potent anti-cytokine agent directly to the site of action of respiratory disease in the lung while limiting systemic exposure. Such lung-selective JAK inhibitors are known in the art and include, for example, TD-8236 (which is disclosed in U.S. Pat. No. 10,100,049).

Compound 1 has been designed for the treatment of inflammatory and fibrotic disease of the respiratory tract. In particular, the compound has been designed to enable delivery of a potent anti-cytokine agent directly to the site of action of respiratory disease in the lung while limiting systemic exposure.

Compound 1 is a potent inhibitor of the JAK family of enzymes: JAK1, JAK2, JAK3, and TYK2, and a potent inhibitor of pro-inflammatory and pro-fibrotic cytokines. It has been recognized that the broad anti-inflammatory effect of systemically available JAK inhibitors could suppress normal immune cell function, potentially leading to an increased risk of infections. By contrast, compound 1 enables the delivery of a potent anti-cytokine agent directly to the site of action of the respiratory disorder, in the lung, while limiting systemic exposure.

Compound 1 was evaluated in a phase 1 clinical trial and dosed in humans through nebulized inhalation at 1 mg, 3 mg, and 10 mg for up to 7 days. The plasma Cₘₐₓ (maximum plasma concentration) values of the compound of formula 1 were found to be well under the binding-corrected JAK IC₅₀, i.e., the plasma concentration necessary to inhibit Janus kinases by 50%. The pharmacokinetics of inhaled compound 1 are consistent with low plasma exposures after inhaled administration. Maximal plasma exposures of compound 1 were about 20-fold and about 7-fold lower than the protein-adjusted JAK IC₅₀ at dose levels of 3 and 10 mg, respectively. Additionally, absolute NK cell counts were evaluated after multiple-dosing to assess the potential for systemic pharmacologic effects associated with JAK inhibition by compound 1. No reductions in NK cells were observed relative to baseline in participants receiving placebo or compound 1 at any dose level (1, 3, or 10 mg) explored in the study. The lack of reduction in NK cell counts is also consistent with the lack of systemic JAK inhibition. In contrast, marked reductions in NK cell counts have been observed with systemic JAK inhibitors such as tofacitinib (Weinhold, K. J., et al., Reversibility of peripheral blood leukocyte phenotypic and functional changes after exposure to and withdrawal from tofacitinib, a Janus kinase inhibitor, in healthy volunteers. Clin Immunol. 191, 10-20, 2018). Other systemically mediated hematological changes associated with JAK inhibition, including neutrophil and hemoglobin reductions as well as lipid changes, were not observed with inhaled administration of compound 1. These results support a favorable safety and tolerability profile and PK below levels anticipated to exert systemic effects.

The anti-inflammatory activity of JAK inhibitors has been robustly demonstrated in preclinical models of asthma (Malaviya et al., Int. Immunopharmacol., 2010, 10, 829-836; Matsunaga et al., Biochem. and Biophys. Res. Commun., 2011, 404, 261-267; Kudlacz et al., Eur. J. Pharmacol, 2008, 582, 154-161). Accordingly, JAK inhibitors, such as lung-selective JAK inhibitors as described herein, are expected to be useful for the treatment of inflammatory respiratory disorders, such as asthma.

Cytokines implicated in asthma inflammation which signal through the JAK-STAT pathway include IL-2, IL-3, IL-4, IL-5, IL-6, IL-9, IL-11, IL-13, IL-23, IL-31, IL-27, thymic stromal lymphopoietin (TSLP), interferon-γ (IFNγ) and granulocyte-macrophage colony-stimulating factor (GM-CSF). Accordingly, compound 1 is expected to be useful for the treatment of inflammatory respiratory disorders, in particular, asthma. Asthma has been classified as Th2 low and Th2 high subtypes (Simpson et al, Respirology, 2006, 11, 54-61). IL-4, IL-13, IL-5, and TSLP are implicated in Th2 high asthma, while IL-23/IL-12, IL-6, IL-27, and IFNγ are implicated in Th2 low asthma. Based on its pan JAK inhibitory profile, compound 1 potently inhibits mediators of both Th2 high and Th2 low asthma. It is therefore expected that compound 1 will be useful in the treatment of both Th2 high and Th2 low asthma.

Inflammation and fibrosis of the lung is characteristic of other respiratory diseases in addition to asthma such as chronic obstructive pulmonary disease (COPD), cystic fibrosis (CF), pneumonitis, interstitial lung diseases (including idiopathic pulmonary fibrosis), acute lung injury, acute respiratory distress syndrome, bronchitis, emphysema, bronchiolitis obliterans, and sarcoidosis. JAK inhibitors, such as lung-selective JAK inhibitors as described herein as well as Compound 1, therefore, are also expected to be useful for the treatment of chronic obstructive pulmonary disease, cystic fibrosis, pneumonitis, interstitial lung diseases (including idiopathic pulmonary fibrosis), acute lung injury, acute respiratory distress syndrome, bronchitis, emphysema, bronchiolitis obliterans, and sarcoidosis.

When compared to its corresponding fluorinated analog, compound 1 has been shown to have similar JAK activity. However, it has the advantage of giving rise to significantly less sulfation metabolism, as demonstrated in the assay section. This is significant as sulfation metabolism occurs in the lungs, which could lead to a rapid decrease in exposure of the active parent compound.

Compound 1 has demonstrated inhibition of cytokines associated with inflammation. Therefore, compound 1 is likely to be useful for the treatment of certain specific respiratory diseases and disorders, as detailed below.

Eosinophilic airway inflammation is a characteristic feature of diseases collectively termed eosinophilic lung diseases (Cottin et al., Clin. Chest. Med., 2016, 37(3), 535-56). Eosinophilic diseases have been associated with IL-4, IL-13 and IL-5 signaling. Eosinophilic lung diseases include infections (especially helminthic infections), drug-induced pneumonitis (induced for example by therapeutic drugs such as antibiotics, phenytoin, or 1-tryptophan), fungal-induced pneumonitis (e.g. allergic bronchopulmonary aspergillosis), hypersensitivity pneumonitis and eosinophilic granulomatosis with polyangiitis (formerly known as Churg-Strauss syndrome). Eosinophilic lung diseases of unknown etiology include idiopathic acute eosinophilic pneumonia, idiopathic chronic eosinophilic pneumonia, hypereosinophilic syndrome, and Löffler's syndrome.

A polymorphism in the IL-6 gene has been associated with elevated IL-6 levels and an increased risk of developing pulmonary arterial hypertension (PAH) (Fang et al., J. Am. Soc. Hypertens., 2017, 11(3), 171-177). Corroborating the role of IL-6 in PAH, inhibition of the IL-6 receptor chain gp130 ameliorated the disease in a rat model of PAH (Huang et al., Can. J. Cardiol., 2016, 32(11), 1356.e1-1356.e10).

Cytokines such as IFNγ, IL-12 and IL-6 have been implicated in a range of non-allergic lung diseases such as sarcoidosis, and lymphangioleiomyomatosis (El-Hashemite et al., Am. J. Respir. Cell. Mol. Biol., 2005, 33, 227-230, and El-Hashemite et al., Cancer Res., 2004, 64, 3436-3443). The compound of the disclosure has also been shown to inhibit IL-6 and IFNγ signaling.

Bronchiectasis and infiltrative pulmonary diseases are diseases associated with chronic neutrophilic inflammation.

Pathological T cell activation is critical in the etiology of multiple respiratory diseases. Autoreactive T cells play a role in bronchiolitis obliterans organizing pneumonia (also termed COS). Similar to COS, the etiology of lung transplant rejections is linked to an aberrant T cell activation of the recipients T cells by the transplanted donor lung. Lung transplant rejections may occur early as Primary Graft Dysfunction (PGD), organizing pneumonia (OP), acute rejection (AR) or lymphocytic bronchiolitis (LB) or they may occur years after lung transplantation as Chronic Lung Allograft Dysfunction (CLAD). CLAD was previously known as bronchiolitis obliterans (BO) but now is considered a syndrome that can have different pathological manifestations including BO, restrictive CLAD (rCLAD or RAS) and neutrophilic allograft dysfunction. Chronic lung allograft dysfunction (CLAD) is a major challenge in long-term management of lung transplant recipients as it causes a transplanted lung to progressively lose functionality (Gauthier et al., Curr Transplant Rep., 2016, 3(3), 185-191). CLAD is poorly responsive to treatment and therefore, there remains a need for effective compounds capable of preventing or treating this condition. Several JAK-dependent cytokines such as IFNγ and IL-5 are up-regulated in CLAD and lung transplant rejection (Berastegui et al, Clin. Transplant. 2017, 31, e12898). Moreover, high lung levels of CXCR3 chemokines such as CXCL9 and CXCL10 which are downstream of JAK-dependent IFN signaling, are linked to worse outcomes in lung transplant patients (Shino et al, PLOS One, 2017, 12 (7), e0180281). Systemic JAK inhibition has been shown to be effective in kidney transplant rejection (Vicenti et al., American Journal of Transplantation, 2012, 12, 2446-56). Therefore, JAK inhibitors have the potential to be effective in preventing or delaying lung transplant rejection and CLAD. Similar T cell activation events as described as the basis for lung transplant rejection also are considered the main driver of lung graft-versus-host disease (GVHD) which can occur post hematopoietic stem cell transplants. Similar to CLAD, lung GVHD is a chronic progressive condition with extremely poor outcomes and no treatments are currently approved. A retrospective, multicenter survey study of 95 patients with steroid-refractory acute or chronic GVHD who received the systemic JAK inhibitor ruxolitinib as salvage therapy demonstrated complete or partial response to ruxolitinib in the majority of patients including those with lung GVHD (Zeiser et al, Leukemia, 2015, 29, 10, 2062-68). As systemic JAK inhibition is associated with serious adverse events and a small therapeutic index, the need remains for an inhaled lung-directed, non-systemic JAK inhibitor to prevent or delay lung transplant rejection or lung GVHD. Compound 1 has the characteristics required to meet this need. More recently, immune-checkpoint inhibitor induced pneumonitis, another T cell mediated lung disease emerged with the increased use of immune-checkpoint inhibitors. In cancer patients treated with these T cell stimulating agents, fatal pneumonitis can develop.

The mixed lymphocyte reaction assay is an in-vitro assay that mimics transplant rejection. Compound 1 was shown to effectively inhibit IFNγ secretion.

In some embodiments, therefore, the disclosure provides a method for treating a respiratory disorder in a subject in need thereof when the subject is receiving high-flow oxygen through a nasal cannula, the method comprising administering a pharmaceutical composition comprising compound 1, or a pharmaceutically acceptable salt thereof, to the subject by oral nebulized inhalation while the subject is receiving high-flow oxygen through a nasal cannula at a rate greater than or equal to about 30 L/min.

In some embodiments, the respiratory disorder is selected from the group consisting of a coronavirus infection, lung transplant rejection, asthma, chronic obstructive pulmonary disease, cystic fibrosis, pneumonitis, idiopathic pulmonary fibrosis, acute lung injury, acute respiratory distress syndrome, bronchitis, emphysema, bronchiolitis obliterans, sarcoidosis, an eosinophilic disease, a helminthic infection, pulmonary arterial hypertension, lymphangioleiomyomatosis, bronchiectasis, an infiltrative pulmonary disease, drug-induced pneumonitis, fungal induced pneumonitis, allergic bronchopulmonary aspergillosis, hypersensitivity pneumonitis, eosinophilic granulomatosis with polyangiitis, idiopathic acute eosinophilic pneumonia, idiopathic chronic eosinophilic pneumonia, hypereosinophilic syndrome, Löffler's syndrome, bronchiolitis obliterans organizing pneumonia, lung graft-versus-host disease, and immune-checkpoint-inhibitor induced pneumonitis. In some embodiments, the respiratory disorder is asthma or chronic obstructive pulmonary disease. In some embodiments, the respiratory disorder is asthma. In some embodiments, the asthma is moderate to severe asthma. In some embodiments, the asthma is mild to moderate asthma. In some embodiments, the asthma is Th2 high asthma. In some embodiments, the asthma is Th2 low asthma. In some embodiments, the respiratory disorder is chronic obstructive pulmonary disease. In some embodiments, the respiratory disorder is lung transplant rejection. In some embodiments, the respiratory disorder is a coronavirus infection.

In some embodiments, the disclosure provides a method of preventing or delaying lung transplant rejection in a subject in need thereof when the subject is receiving high-flow oxygen through a nasal cannula, the method comprising administering a pharmaceutical composition comprising compound 1, or a pharmaceutically acceptable salt thereof, to the subject by oral nebulized inhalation while the subject is receiving high-flow oxygen through a nasal cannula at a rate greater than or equal to about 30 L/min. In some embodiments, the lung transplant rejection is selected from the group consisting of primary graft dysfunction, organizing pneumonia, acute rejection, lymphocytic bronchiolitis, and chronic lung allograft dysfunction. In some embodiments, the lung transplant rejection is acute lung transplant rejection. In some embodiments, the lung transplant rejection is chronic lung allograft dysfunction. In some embodiments, the lung transplant rejection is selected from the group consisting of bronchiolitis obliterans, restrictive chronic lung allograft dysfunction, and neutrophilic allograft dysfunction. In some embodiments, the pharmaceutical composition is administered by inhalation.

The disclosure further provides a method of treating asthma in a subject in need thereof when the subject is receiving high-flow oxygen through a nasal cannula, the method comprising administering a pharmaceutical composition comprising compound 1, or a pharmaceutically acceptable salt thereof, to the subject by oral nebulized inhalation while the subject is receiving high-flow oxygen through a nasal cannula at a rate greater than or equal to about 30 L/min.

When used to treat asthma, compound 1 will typically be administered in a single daily dose or in multiple doses per day, although other forms of administration may be used. The amount of active agent administered per dose or the total amount administered per day will typically be determined by a physician, in the light of the relevant circumstances, including the condition to be treated, the chosen route of administration, the age, weight, and response of the individual subject, the severity of the subject's symptoms, and the like.

The disclosure further provides a method of treating a respiratory disorder (including but not limited to the disorders described herein) in a subject in need thereof when the subject is receiving high-flow oxygen through a nasal cannula, the method comprising administering a pharmaceutical composition comprising compound 1, or a pharmaceutically acceptable salt thereof, to the subject by oral nebulized inhalation while the subject is receiving high-flow oxygen through a nasal cannula at a rate greater than or equal to about 30 L/min.

When used to treat a respiratory disorder (including but not limited to the disorders described herein), compound 1 will typically be administered in a single daily dose or in multiple doses per day, although other forms of administration may be used. The amount of active agent administered per dose or the total amount administered per day will typically be determined by a physician, in the light of the relevant circumstances, including the condition to be treated, the chosen route of administration, the age, weight, and response of the individual subject, the severity of the subject's symptoms, and the like.

Human coronavirus is a common respiratory pathogen and typically induces mild upper respiratory disease. The two highly pathogenic viruses, Severe Acute Respiratory Syndrome associated-Coronavirus (SARS-CoV-1) and Middle East Respiratory Syndrome-associated Coronavirus (MERS-CoV), caused severe respiratory syndromes resulting in more than 10% and 35% mortality, respectively (Assiri et al., N Engl J Med., 2013, 369, 407-1). The recent emergence of Coronavirus Disease 2019 (COVID-19) and the associated pandemic has created a global health care emergency. Similar to SARS-CoV-1 and MERS-CoV, a subset of patients (about 16%) can develop a severe respiratory illness manifested by acute lung injury (ALI) leading to ICU admission (about 5%), respiratory failure (about 6.1%) and death (Wang et al., JAMA, 2020, 323, 11, 1061-1069; Guan et al., N Engl J Med., 2020, 382, 1708-1720; Huang et al., The Lancet, 2020. 395 (10223), 497-506; Chen et al., The Lancet, 2020, 395(10223), 507-13). A subgroup of patients with COVID-19 appears to have a hyperinflammatory “cytokine storm” resulting in acute lung injury and acute respiratory distress syndrome (ARDS). This cytokine storm may also spill over into the systemic circulation and produce sepsis and ultimately, multi-organ dysfunction syndrome. The dysregulated cytokine signaling that appears in COVID-19 is characterized by increased expression of interferons (IFNs), interleukins (ILs), and chemokines, resulting in ALI and associated mortality.

Infection with mouse adapted strains of the 2003 SARS-CoV-1 and 2012 MERS-CoV, as well as a transgenic mouse expressing the human SARS-CoV-1 receptor hACE2 infected with human SARS-CoV-1, demonstrate elevations of JAK-dependent cytokines, such as IFNγ, IL-6, and IL-12, and downstream chemokines, such as chemokine (C—C motif) ligand 10 (CCL10), CCL2, and CCL7 (McCray et al., J Virol., 2007, 81(2), 813-21; Gretebeck et al., Curr Opin Virol. 2015, 13, 123-9; Day et al., Virology. 2009, 395(2), 210-22. It was recently shown that similar to SARS-CoV-1 and MERS-CoV, patients with severe COVID-19 have elevated Th17, which can be driven by IL-6 and IL-23 via signal transducer and activator of transcription 3, STAT3 (Huang et al., Lancet 2020, 395, 497-506). Mouse Th17 cells produce large amounts of IL-17 in response to IL-23 which can be blocked with a JAK inhibitor (Wu et al., J Microbiol Immunol Infect., 2020, S1684118220300657). Though IFN responses can be protective in virus infection, there is evidence that a delayed response in humans contributes to virus-induced acute respiratory distress syndrome (Chen et al., Annu Rev Immunol., 2007, 25(1), 443-72). Similarly, mice deficient in the IFNα/β receptor IFNR1 are protected from lethal SARS-CoV-1 infection (Channappanavar R, Fehr A R, Vijay R, Mack M, Zhao J, Meyerholz D K, et al. Dysregulated Type I Interferon).

Concerns have also been raised about the potential increased risk for thromboembolism with systemic JAK inhibitors which is being particularly concerning given observations of severe hypercoagulability in patients with COVID-19.

Compound 1, a lung-selective, inhaled pan-JAK inhibitor addresses the shortcomings of oral JAK inhibitors by avoiding systemic immunosuppression, thromboembolisms, and additional infections that lead to worsened mortality.

Further, compound 1 has been used and can be used alone or in combination with standard of care including remdesivir and corticosteroids such as dexamethasone.

Compound 1 acts through a mechanism of action that can dampen the cytokine storm associated with a coronavirus infection.

Compound 1 has been studied in a phase 2 clinical study in subjects infected with COVID. Although there was no statistically significant difference in RFDs from randomization through Day 28 between compound 1 and placebo in ITT (median: 21 vs. 21 days; p=0.61) and no difference in change from baseline at Day 7 in SaO₂/FiO₂ ratio, proportion of subjects in each category of the 8-point Clinical Status scale, and proportion of subjects alive and respiratory failure-free at Day 28, compound 1 demonstrated a favorable trend in improvement when compared to placebo for 28-day all-cause mortality (total number of deaths: 6 vs. 13, HR: 0.42, p=0.08) and time to recovery (median: 10 vs. 11 days, HR: 1.27, p=0.12).

Importantly, in a post-hoc analysis of subjects with baseline CRP (n=201), in subjects with CRP <150 mg/L (n=171), there was an improvement in those treated with compound 1 when compared to placebo in:
28-day all-cause mortality (total number of deaths: 1 vs 9, HR: 0.097, p=0.009).
time to recovery (median: 10 vs. 11 days, HR: 1.48, p=0.02).

In subjects with CRP >150 mg/L (n=30), there was no difference in time to recovery or 28-day all-cause mortality between those treated with compound 1 or placebo.

Therefore, compound 1 is particularly adapted for the treatment of the subpopulation of subjects having a CRP (C-reactive protein) baseline level below 150 mg/L. In some embodiments, the subject has a baseline level of C-reactive protein less than or equal to about 150 mg/L.

C-reactive protein (CRP) is a protein found in blood plasma, whose circulating concentrations rise in response to inflammation. It is an acute-phase protein of hepatic origin that increases following interleukin-6 secretion by macrophages and T cells. In healthy adults, the normal concentrations of CRP varies between 0.8 mg/L and 3.0 mg/L. However, some healthy adults show elevated CRP at 10 mg/L.

Compound 1 was well-tolerated. Adverse events and serious adverse events occurred in 34.0% and 9.7% of subjects treated with compound 1, and 41.2% and 15.7% of subjects treated with placebo, respectively. Adverse events of liver abnormalities or disease occurred in 9.7% and 7.8% of subjects treated with compound 1 and placebo, respectively. Serious infections and venous thromboembolism occurred in 1.0% and none of the subjects treated with compound 1, and 2.0% and 4.9% in subjects treated with placebo, respectively.

Plasma exposure of compound 1 was low and consistent with expectations for a lung-selective medicine.

Additionally, coronaviruses gain entry into host cells by fusing with cellular membranes, a step that is required for virus replication. Abelson kinase inhibitors have been reported to be potent inhibitors of SARS-CoV-1 and MERS-CoV fusion (Coleman et al., Journal of Virology, 2016, 90, 19, 8924-8933; Sisk et al., Journal of General Virology, 2018, 99, 619-630), supporting the fact that an Abelson kinase inhibitor could be useful for treating patients infected with a coronavirus by decreasing the viral load of the patient. Compound 1 has been shown to potently inhibit Abl2.

Therefore, without being limited by this theory, compound 1 may be uniquely suited for the treatment of coronaviruses, as a compound which can be delivered selectively to the lungs, possesses pan-JAK inhibitory activity that can dampen the cytokine storm associated with COVID-19, and possesses Abelson kinase inhibitory activity that could decrease the viral load of the coronavirus in the subject.

In some embodiments, the disclosure provides a method for treating a SARS-CoV-2 lung infection in a subject in need thereof when the subject is receiving high-flow oxygen through a nasal cannula, the method comprising administering a pharmaceutical composition comprising nezulcitinib or a pharmaceutically acceptable salt thereof, to the subject by oral nebulized inhalation while the subject is receiving high-flow oxygen through a nasal cannula at a rate greater than or equal to about 30 L/min.

Abl kinases have also been reported to have a positive role in regulating endothelial barrier function and vascular leak during acute lung injury. Therefore, compound 1, or a pharmaceutically acceptable salt thereof, may also work by strengthening endothelial cell-to-cell contacts and promoting endothelial cell adhesion to the extracellular matrix.

Under another theory, the potential ability of compound 1 to directly affect the coronavirus may counter-balance or mitigate a possible increase in local viral replication caused by the use of a compound causing immune suppression.

It has also been reported that the ability of neutrophils to form neutrophil extracellular traps (NETs) may contribute to organ damage and mortality in COVID-19 patients (Barnes et al., J. Exp. Med., 2020, 217, 6, e20200652, 1-7). Aberrant NET formation has been linked to pulmonary diseases, thrombosis, mucous secretions in the airways, and cytokine production. Therefore, compound 1, or a pharmaceutically acceptable salt thereof, may be useful to (a) block or inhibit neutrophilia and/or the formation of neutrophil extracellular traps (NETs) in a subject infected with a coronavirus, (b) decrease the risk of thrombosis in a subject infected with a coronavirus, and/or (c) decrease the incidence of thrombosis in a subject population infected with a coronavirus.

Multisystem inflammatory syndrome in children (MIS-C) is a condition where different body parts can become inflamed, including the heart, lungs, kidneys, brain, skin, eyes, or gastrointestinal organs. MIS-C has been associated with exposure to COVID-19 and appears to be a rare but serious complication associated with COVID-19. MIS-C is associated with inflammation of the lungs. Therefore, compound 1 is expected to be useful in preventing or treating MIS-C.

Further, respiratory epithelial cell death by influenza virus infection is responsible for the induction of inflammatory responses. It has been shown that Influenza A virus infection triggers pyroptosis and apoptosis of respiratory epithelial cells through the Type I Interferon signaling pathway (Lee et al., Journal of Virology, 2018, 92, 14, e00396-18). The type I interferon (IFN)-mediated JAK-STAT signaling pathway promotes the switch from apoptosis to pyroptosis by inhibiting apoptosis possibly through the induced expression of the Bcl-xL anti-apoptotic gene. Further, the inhibition of JAK-STAT signaling repressed pyroptosis but enhanced apoptosis in infected PL16T cells. This suggests that the type I IFN signaling pathway plays an important role to induce pyroptosis but represses apoptosis in the respiratory epithelial cells to initiate proinflammatory responses against influenza virus infection. Accordingly, the compound of formula 1, or a pharmaceutically acceptable salt thereof, is expected to be useful to treat subjects infected with influenza. Based on its mechanism of action, the compound of formula 1, or a pharmaceutically acceptable salt thereof, is expected to prevent or treat inflammation in the lungs and/or ALI and/or ARDS in subjects infected with influenza.

Also provided herein are uses of compound 1 in medical therapy and in the manufacture of a formulation or medicament for treating, preventing, delaying, or ameliorating diseases and disorders amenable to treatment with a JAK inhibitor, in particular respiratory disorders, lung transplant rejection, and coronavirus infections.

### Combination Therapy

In some embodiments, the methods described herein comprise administering one or more additional therapeutic agents or treatments to the subject. Compound 1, or a pharmaceutically acceptable salt thereof, may be used in combination with one or more additional therapeutic agents or treatments which act by the same mechanism or by different mechanisms to treat a disease. The different therapeutic agents or treatments may be administered sequentially or simultaneously, in separate compositions or in the same composition. Useful classes of therapeutical agents for combination therapy include, but are not limited to, an IL-6 inhibitor, an IL-6 receptor antagonist, an IL-6 receptor agonist, an IL-2 inhibitor, an antiviral, an anti-inflammatory drug, a sodium-glucose cotransporter 2 inhibitor, a vaccine, an ACE2 inhibitor, an antibiotic, an antiparasitic, a sphingosine 1-phosphate receptor modulator, a TMPRSS2 inhibitor, a TNF alpha inhibitor, an anti-TNF, a membrane haemagglutinin fusion inhibitor, an inhibitor of the terminal glycosylation of ACE2, a CCR5 inhibitor, stem cells, allogeneic mesenchymal stem cells, CRISPR therapy, CAR-T therapy, TCR-T therapy, a virus-neutralizing monoclonal antibody, a protease inhibitor, a SARS-CoV-2 antibody, a siRNA, a plasma-derived immunoglobulin therapy, a S-protein modulator, a PLX stem cell therapy, chimeric humanized virus suppressing factor, multipotent adult progenitor cell therapy, an anti-viroporin, umbilical cord-derived mesenchymal stem cells, a polymerase inhibitor, autologous adipose-derived mesenchymal stem cells, an angiotensin converting enzyme 2 inhibitor, an immunoglobulin agonist, a nucleoside reverse transcriptase inhibitor, a cytotoxic T-lymphocyte protein-4 inhibitor, a lung surfactant associated protein D modulator, a protease inhibitor, a nuclear factor kappa B inhibitor, a xanthine oxidase inhibitor, an endoplasmin modulator, a CCL26 gene inhibitor, a TLR modulator, a TLR agonist, a TLR-2 agonist, a TLR-6 agonist, a TLR-9 agonist, a TLR-4 agonist, a TLR-7 agonist, a TLR-3 agonist, an opioid receptor antagonist, a moesin inhibitor, an angiotensin converting enzyme 2 modulator, a MEK protein kinase inhibitor, aCD40 ligand receptor agonist, a CD70 antigen modulator, an amyloid protein deposition inhibitor, an apolipoprotein gene stimulator, a bromodomain containing protein 2 inhibitor, a bromodomain containing protein 4 inhibitor, an IL-15 receptor agonist, an immunoglobulin gamma Fc receptor III agonist, a MEK-1 protein kinase inhibitor, a Ras gene inhibitor, an interferon beta ligand, a galectin-3 inhibitor, a heat shock protein inhibitor, an elongation factor 1 alpha 2 modulator, a VEGF-1 receptor modulator, an Angiotensin II AT-2 receptor agonist, a basigin inhibitor, a viral envelope glycoprotein inhibitor, a gelsolin stimulator, a trypsin inhibitor, a GM-CSF ligand inhibitor, a urokinase plasminogen activator inhibitor, a serine protease inhibitor, a PDE 3 inhibitor, a PDE 4 inhibitor, a C-reactive protein inhibitor, a chemokine CC22 ligand inhibitor, a GM-CSF receptor antagonist, an hemoglobin scavenger receptor antagonist, a metalloprotease-1 inhibitor, a metalloprotease-3 inhibitor, a metalloprotease inhibitor, a small inducible cytokine A17 ligand inhibitor, a VEGF gene inhibitor, a Coronavirus spike glycoprotein inhibitor, a nucleoprotein inhibitor, an ATP binding cassette transporter B5 modulator, a vimentin modulator, a stem cell antigen-1 inhibitor, a casein kinase II inhibitor, a complement C5a factor inhibitor, an aldose reductase inhibitor, a calpain-I inhibitor, a calpain-II inhibitor, a calpain-IX inhibitor, a proto-oncogene Mas agonist, a non-nucleoside reverse transcriptase inhibitor, an Interferon gamma ligand inhibitor, a CD4 modulator, a TGFB2 gene inhibitor, an Interleukin-1 beta ligand inhibitor, an inosine monophosphate dehydrogenase inhibitor, an angiotensin converting enzyme 2 stimulator, an adenosine A3 receptor agonist, a palmitoyl protein thioesterase 1 inhibitor, a Btk tyrosine kinase inhibitor, a NK1 receptor antagonist, an acetaldehyde dehydrogenase inhibitor, a CGRP receptor antagonist, a prostaglandin E synthase-1 inhibitor, a VIP receptor agonist, a nuclear factor kappa B gene modulator, a Grp78 calcium binding protein inhibitor, a Jun N terminal kinase inhibitor, a transferrin modulator, a p38 MAP kinase modulator, a CCR5 chemokine antagonist, a APOA1 gene stimulator, a bromodomain containing protein 2 inhibitor, a bromodomain containing protein 4 inhibitor, a BMP10 gene inhibitor, a BMP15 gene inhibitor, an adrenergic receptor antagonist, a human papillomavirus E6 protein modulator, a human papillomavirus E7 protein modulator, a Ca2+ release activated Ca2+ channel 1 inhibitor, an amyloid protein deposition inhibitor, a gamma-secretase inhibitor, a 2,5-Oligoadenylate synthetase stimulator, an Interferon type I receptor agonist, a ribonuclease stimulator, a S phase kinase associated protein 2 inhibitor, a dehydropeptidase-1 modulator, a calcium channel modulator, a signal transducer CD24 modulator, a cyclin E inhibitor, a cyclin-dependent kinase-2 inhibitor, a cyclin-dependent kinase-5 inhibitor, a cyclin-dependent kinase-9 inhibitor, a GM-CSF ligand inhibitor, an Interferon receptor modulator, an Interleukin-29 ligand, a cyclin-dependent kinase-7 inhibitor, a MCL1 gene inhibitor, a complement C5 factor inhibitor, an heparin agonist, an exo-alpha sialidase modulator, a muscarinic receptor antagonist, an IL-8 receptor antagonist, a vitamin D3 receptor agonist, a high mobility group protein B1 inhibitor, a CASP8-FADD-like regulator inhibitor, an ecto NOX disulfide thiol exchanger 2 inhibitor, a sphingosine kinase inhibitor, a sphingosine-1-phosphate receptor-1 antagonist, a stimulator of interferon genes protein stimulator, a topoisomerase inhibitor, an X-linked inhibitor of apoptosis protein inhibitor, an angiopoietin ligand-2 inhibitor, a neuropilin 2 inhibitor, a listeriolysin stimulator, an Interferon gamma receptor agonist, a MAPK gene modulator, a GM-CSF ligand inhibitor, an immunoglobulin G1 modulator, an immunoglobulin kappa modulator, a kallikrein modulator, a mannan-binding lectin serine protease inhibitor, an ubiquitin modulator, an IL12 gene stimulator, a xanthine oxidase inhibitor, a dihydroorotate dehydrogenase inhibitor, an IL-17 antagonist, a MAP kinase inhibitor, a PARP inhibitor, a poly ADP ribose polymerase 1 inhibitor, a poly ADP ribose polymerase 2 inhibitor, a dipeptidyl peptidase I inhibitor, a Btk tyrosine kinase inhibitor, a type I IL-1 receptor antagonist, an exportin 1 inhibitor, a hyaluronidase inhibitor, a sodium glucose transporter-2 inhibitor, a dihydroceramide delta 4 desaturase inhibitor, a sphingosine kinase 2 inhibitor, an Interferon beta ligand, an ICAM-1 stimulator, a TNF antagonist, a vascular cell adhesion protein 1 agonist, a COVID19 Spike glycoprotein modulator, a complement CIs subcomponent inhibitor, a NMDA receptor epsilon 2 subunit inhibitor, a tankyrase-1 inhibitor, a protein translation initiation inhibitor, a sigma receptor modulator, a sigmaR1 receptor modulator, a sigmaR2 receptor modulator, an antihistamine, an anti-C5aR, a RNAi. a corticosteroid, a BCR-ABL a tyrosine kinase inhibitor, a colony stimulating factor, an inhibitor of tissue factor (TF), a recombinant granulocyte macrophage colony-stimulating factor (GM-CSF), a Gardos channel blocker, a heat-shock protein 90 (Hsp90) inhibitor, an alpha blocker, a cap binding complex modulator, a LSD1 inhibitor, a CRAC channel inhibitor, a RNA polymerase inhibitor, a CCR2 antagonist, a DHODH inhibitor, a blood thinner, an anti-coagulant, a factor Xa inhibitor, a SSRI, a SNRI, a sigma-1 receptor activator, a beta-blocker, a caspase inhibitor, a serine protease inhibitor, an IL-23A modulator, a NLRP3 inhibitor, an Angiopoietin-Tie2 signaling pathway modulator, a mannan-binding lectin-associated serine protease-2 modulator, a PDE4 inhibitor, a Vasoactive Intestinal Polypeptide, a microtubule depolymerization agent, a (PD)-1 checkpoint inhibitor, an Axl kinase inhibitor, a (PD)-1/PD-L1 checkpoint inhibitor, a PD-L1 checkpoint inhibitor, a T-cell CD61 receptor modulator, a Factor XIIa antagonist, an oral spleen tyrosine kinase (SYK) inhibitor, a CK2 inhibitor, a NMDA receptor antagonist, a SK2 inhibitor, an antiandrogen, and a tankyrase-2 inhibitor.

Specific therapeutic agents that may be used in combination with compound 1 include, but are not limited to cidofovir triphosphate, cidofovir, abacavir, ganciclovir, stavudine triphosphate, 2′-O-methylated UTP, desidustat, ampion, trans sodium crocetinate, CT-P59, Ab8, heparin, apixaban, GC373, GC376, Oleandrin, GS-441524, sertraline, Lanadelumab, zilucoplan, abatacept, CLBS119, Ranitidine, Risankizumab, AR-711, AR-701, MP0423, bempegaldesleukin, melatonin, carvedilol, mercaptopurine, paroxetine, casirivimab, imdevimab, ADG20, emricasan, dapansutrile, ceniciviroc infliximab, DWRX2003, AZD7442, MAN-19, LAU-7b, niclosamide, ANA001, fluvoxamine, narsoplimab, Sarconeos, GIGA-2050, VERU-111, REGN-COV2, icatibant, cenicriviroc, NTR-441, LAM-002A, oseltamivir, VHH72-Fc, MK-4482, EB05, OB-002, CM-4620-IE, IMU-838, SNG001, NT-17, BOLD-100, WP1122, itolizumab, PB1046, fostamatinib, colchicine, M5049, EDP1815, ABX464, CPI-006, azelastine, garadacimab, silmitasertib, lopinavir, ritonavir, remdesivir, cloroquine, hydrochloroquine, convalescent plasma transfusion, azithromycin, tocilizumab, famotidine, sarilumab, interferon beta, interferon beta-Ia, interferon beta-1b, peginterferon lambda-Ia, favipiravir, ASDC-09, dapagliflozin, CD24Fc, ribavirin, umifenovir, nitric oxide, APNO1, teicoplanin, oritavancin, dalbavancin, monensin, ivermectin, darunavir, cobicistat, fingolimod, camostat, galidesicir, thalomide, leronlimab, remestemcel-L, canakinumab, TAK-888, azvudine, BPI-002, AT-100, T-89, Neumifil, GreMERSfi, liposomal curcumin, OYA-1, oxypurinol, mosedipimod, PUL-042, naltrexone, metenkefalin, COVID-EIG, TNX-1800, ATR-002, 177Lu-EC-Amifostine, 99mTc-EC-Amifostine, apabetalone, STI-6991, STI-4398, antroquinonol, ZIP-1642, DPX-COVID-19, belapectin, GX-19, AdCOVID, siltuximab, IBIO-200, plitidepsin, C-21, meplazumab, pathogen-specific aAPC, LV-SMENP-DC, ARMS-I, rhu-pGSN, PRTX-007, CK-0802, namilumab, upamostat, NI-007, COVID-HIG, CYNK-001, Nafamostat, brilacidin, mavrilimumab, IPT-001, PittCoVacc, allo-APZ2-Covid19, ENU-200, VIR-7832, VIR-7831, pritumumab, Ampion, TZLS-501, sodium pyruvate, silmitasertib, CoroFlu, BDB-1, AT-001, BLD-2660, 20-hydroxyecdysone, IFX-1, elsulfavirine, emapalumab, CEL-1000, trabedersen, VBI-2901, ASC-09, TJM-2, RPH-104, tranexamic acid, WP-1122, olokizumab, APN-O1, danoprevir, piclidenoson, FW-1022, CORAVAX, Lamellasome COVID-19, COVID-19 XWG-03, EIDD-2801, AVM-0703, DC-661, acalabrutinib, bitespiramycin, Allocetra, tradipitant, bacTRL-Tri, Ad5-nCoV, EPV-CoV19, ADX-629, vazegepant, mercaptamine, sonlicromanol, aviptadil, fenretinide, IT-139, nitazoxanide, apabetalone, lucinactant, bacTRL-Spike, SAB-185, NVX-CoV2373, CM-4620, INO-4800, eicosapentaenoic acid, itanapraced, rintatolimod, XAV-19, niclosamide, ciclesonide, DAS181, ORBCEL-C, Metablok, dantrolene, CD24-IgFc, fadraciclib, gimsilumab, seliciclib, Cyto-MSC, ST-266, MRx-0004, ravulizumab, tafoxiparin, DAS-181, BMS-986253, cholecalciferol, nafamostat, ChAdOxi nCoV-19, idronoxil, LY-3127804, ATYR-1923, VPM-1002, Mycobacterium w, lenzilumab, Polyoxidonium, conestat alfa, ubiquitin proteasome modulator, COVID-19 virus main protease Mpro inhibitor, mRNA-1273, clevudine, bucillamine, sodium meta-arsenite, vidofludimus, DARPin, COV-ENT-1, KTH-222, mefuparib, brensocatib, zanubrutinib, anakinra, selinexor, sarilumab, astodrimer, dapagliflozin propanediol, opaganib, BNT-162c2, BNT-162b2, BNT-162b1, BNT-162a1, ifenprodil, PIC1-01, 2X-121, zotatifin, aplidin, cloperastine, clemastine, dociparstat, avdoralimab, VIR-2703, ALN-COV, intravenous immunoglobulin (IVIg), apremilast, vicromax, baloxavir marboxil, emtricitabine, tenofovir, novaferon, secukiunab, valsartan, niatinib, omailzumab lcucine, sofosbuvir, alovudine, zidovudine, R-107. AB-201, sargram-ostim, LYT-100, senicapoc, fluvoxamine, aspirin, losartan, ADX-1612, ADX-629, sirikumab, otilimab, STI-1499, TR-C19, ABX-464, interferon alpha2b, arbidol, 5309, vafidemstat, AT-527, ibudilast, auxora, bemcentinib, eculizumab, JS016, FSD-201, LY-CoV555, avifavir, OP-101, RLF-100, DMX-200, 47D11, remsima, TYR1923, dexamethasone, EDP-1815, PTC29, rabeximtod, foralunab, budesonide, molnupiravir, ensovibep, dalcetrapib, FSD201, pralatrexate, proxalutamide, clofazimne and merimepodib.

In some embodiments, a JAK inhibitor, or a pharmaceutically acceptable salt thereof, is used in combination with an antiviral and/or a corticosteroid. In some embodiments, compound 1, or a pharmaceutically acceptable salt thereof, is used in combination with an antiviral. In some embodiments, compound 1, or a pharmaceutically acceptable salt thereof, is used in combination with a corticosteroid. In some embodiments, compound 1, or a pharmaceutically acceptable salt thereof, is used in combination with an antiviral and a corticosteroid. In some embodiments, the antiviral is remdesivir. In some embodiments, the antiviral is favipiravir. In some embodiments, the antiviral is molnupiravir. In some embodiments, the antiviral is ritonavir-boosted nirmatrelvir. In some embodiments, the corticosteroid is dexamethasone.

Also provided, herein, is a pharmaceutical composition comprising a JAK inhibitor, or a pharmaceutically acceptable salt thereof, and one or more other therapeutic agents. Also provided, herein, is a pharmaceutical composition comprising compound 1, or a pharmaceutically acceptable salt thereof, and one or more other therapeutic agents. The therapeutic agent may be selected from the class of agents specified above and from the list of specific agents described above. In some embodiments, the pharmaceutical composition is suitable for delivery to the lungs. In some embodiments, the pharmaceutical composition is suitable for inhaled or nebulized administration. In some embodiments, the pharmaceutical composition is a dry powder or a liquid composition.

For all the methods disclosed herein, the methods comprise administering to the subject a JAK inhibitor, or a pharmaceutically acceptable salt thereof, and one or more other therapeutic agents. Further, for all the methods disclosed herein, the methods comprise administering to the subject compound 1, or a pharmaceutically acceptable salt thereof, and one or more other therapeutic agents.

When used in combination therapy, the agents may be formulated in a single pharmaceutical composition, or the agents may be provided in separate compositions that are administered simultaneously or at separate times, by the same or by different routes of administration. Such compositions can be packaged separately or may be packaged together as a kit. The two or more therapeutic agents in the kit may be administered by the same route of administration or by different routes of administration.

### EXAMPLES

### Example 1—Clinical Study: A Phase 1, Open-Label Study to Assess the Pharmacokinetics and Safety of Inhaled Nezulcitinib Administered in the Setting of Supplemental Oxygenation Scenarios in Healthy Participants Study Objectives

Part A: assessed the plasma PK of nezulcitinib following single inhaled doses administered in the setting of various supplemental oxygenation scenarios in healthy participants.

Part B: assessed the safety and tolerability of nezulcitinib following single inhaled doses administered in the setting of various supplemental oxygenation scenarios in healthy participants.

This was a Phase 1, open-label, fixed-sequence, 4-period study to evaluate the PK and safety of single inhaled doses of nezulcitinib, administered under different supplemental oxygenation scenarios.

Supplemental oxygenation is commonly used in hospitalized subjects with ALI due to COVID-19 and may be coadministered with nezulcitinib in a clinical setting. High flow rates of supplemental oxygen are known to lower the delivered dose of inhaled medications. It was thus important to evaluate the safety and PK of inhaled nezulcitinib administered in the setting of various supplemental oxygenation scenarios in healthy participants, in order to inform possible dose adjustments and guide administration instructions in future studies.

### Study Participants

Fourteen healthy, adult male and female participants were enrolled. Participants fulfilled all of the following inclusion criteria:
Healthy, adult, male or female, 18 to 65 years of age, inclusive, at the screening visit.
Pregnancy concerns:
   Female subjects must be either of non-childbearing potential or, if of childbearing potential, subject must not be pregnant or breastfeeding, and must agree to use a highly effective birth control method during the study and through 30 days after the last dose of nezulcitinib.
   Male subjects must agree to use condoms to prevent potential fetal or partner exposure through seminal fluid, in addition to the use of highly effective pregnancy prevention measures with female partners of childbearing potential during the study and through 30 days after the last dose of nezulcitinib.
Body mass index (BMI) ≥18.0 and ≤35.0 kg/m² at the screening visit and weighs at least 50 kg at the screening visit.
Medically healthy with no clinically significant medical history, physical examination, spirometry, laboratory profiles, vital signs, or ECGs at screening and prior to dosing in Period 1, as deemed by the Investigator or designee, including the following:
   Liver function tests (i.e., alanine aminotransferase [ALT], aspartate aminotransferase [AST], alkaline phosphatase [ALP], gamma-glutamyl transferase [GGT], and bilirubin)≤upper limit of normal or deemed medically acceptable at the discretion of the Investigator and Sponsor's Medical Monitor.
   Absolute lymphocyte count within normal range or should be deemed medically acceptable at the discretion of the Investigator and Sponsor's Medical Monitor.
Participants must have a negative QuantiFeron® Test result at Screening.
Forced expiratory volume in 1 second (FEV1) ≥80% predicted at Screening.
Able to understand and accept the use of the nebulizer device(s) and breathing through the supplemental oxygenation required procedures at the screening visit and Day −1 of Period 1.
Understands the study procedures in the informed consent form (ICF), and be willing and able to comply with the protocol.

Participants were free to withdraw from the study at any time for any reason, and could have been withdrawn from the study by the Investigator after adverse events or noncompliance with study criteria (i.e., pregnancy, drug or alcohol use, etc.).

### Duration of Participation for Participants

The length of study participation for each participant was approximately 50 days, as follows: a screening period of up to 28 days before the first dose, followed by up to 4 administration periods consisting of a single dose on Day 1 of each period, with a washout period of at least 5 days between each nezulcitinib dose, and a 7 (±2) day follow-up period after the last administered dose of nezulcitinib.

The washout period was based on the PK of nezulcitinib and not the oxygen supplementation, because it was anticipated that any effects of supplemental oxygen (namely changes to pulse oximetry, respiratory rate, and blood pH) will normalize within minutes after cessation of supplemental oxygen in healthy participants. The washout between doses was considered sufficient to prevent carryover effects of the preceding nezulcitinib administration.

### Study Product

The drug product, nezulcitinib inhalation solution, consisted of 3 mg of nezulcitinib in a sterile aqueous solution containing 10 mM citrate buffer, with 150 mM sodium chloride at pH 4.0. It was a clear, colorless-to-yellow solution. The drug product was manufactured at 3 mg/mL strengths (as free base equivalent). It was supplied in unit dose low density polyethylene (LDPE) vials manufactured by Blow-Fill-Seal (BFS) technology and individually packaged in sealed foil laminate pouches. The Aerogen Solo nebulizer system (manufactured by Aerogen Limited, Ireland) was used to administer the drug product. The unit-dose composition of the drug product formulation (3 mg/l mL) is given in Table 1.

TABLE 1
Composition of Nezulcitinib Inhalation Solution, 3 mg/1 mL

| Material | Grade | Function | Quantity (mg/1 mL) |
|---|---|---|---|
| Nezulcitinib | N/A | Drug Substance | 3 (free base equivalent) |
| Citric Acid, Monohydrate | USP, Ph. Eur | Buffering Agent | 2.1 |
| Sodium Chloride | USP, Ph. Eur | Tonicity Agent | 8.8 |
| Hydrochloric Acid | NF, Ph. Eur | pH Adjustment | q.s. to pH 4.0 ± 0.5 |
| Sodium Hydroxide | NF, Ph. Eur | pH Adjustment | q.s. to pH 4.0 ± 0.5 |
| Water for Injection | USP | Solvent | q.s. to 1 mL |

### Dosage, Dosage Form, Route, and Dose Regimen

Participants received a single inhaled dose of nezulcitinib using a nebulizer device on Day 1 of each administration period, under different supplementary oxygenation conditions and under fasting conditions.

Administrations were as follows:
Administration A (test): 3 mg nezulcitinib delivered by nasal inhalation via the Aerogen Solo integrated with a high-flow nasal cannula circuit delivering supplemental oxygen at a flow rate of 30 L/min.
Administration B (reference): 3 mg nezulcitinib delivered by oral inhalation via the Aerogen Solo coupled with the Aerogen Ultra handheld device, in the presence of supplemental oxygen delivery via low-flow nasal cannula at 6 L/min.
Administration C (test): 3 mg nezulcitinib delivered by oral inhalation via the Aerogen Solo coupled with the Aerogen Ultra handheld device, in the presence of supplemental oxygen delivery via high-flow nasal cannula at a flow rate of 30 L/min.
Administration D1 (test): 3 mg nezulcitinib delivered by oral inhalation via the Aerogen Solo coupled with the Aerogen Ultra handheld device, in the presence of supplemental oxygen delivery via high-flow nasal cannula at a flow rate of 50 L/min.
Administration D2 (test): 9 mg nezulcitinib delivered by oral inhalation via the Aerogen Solo coupled with the Aerogen Ultra handheld device, in the presence of supplemental oxygen delivery via high-flow nasal cannula at a flow rate of 50 L/min.

Hour 0 was defined as the beginning of nezulcitinib inhalation session in each dose administration.

Approximately 5 minutes of supplemental oxygen was administered pre-dose to acclimatize participants to the oxygen method corresponding to the administration period in which they were about to be dosed.

The pharmacy at the CRU provided each dose in individual unit dose containers for each participant and for each study period.

Single and multiple doses of 1, 3, and 10 mg nezulcitinib were found safe and well tolerated in healthy adult participants. Systemic exposure (AUC0-24) and Cmax increased in a dose proportional manner following single and multiple doses at the range of 1 to 10 mg of nezulcitinib. The selected dose of 3 mg in the present study was consistent with the dose in a recently completed Phase 2 study. In addition, the 3 mg dose ensured adequate characterization of the nezulcitinib PK profile. Dose levels up to 9 mg of nezulcitinib were also evaluated (Administration D2). This dose level was within the range of doses tested previously (up to 10 mg of nezulcitinib) in healthy participants and those with COVID-19 in previous clinical studies.

Several factors are known to impact the delivery of inhaled medications administered in the presence of high-flow oxygen therapy, including flow rate of gas and position of the nebulizer device within the high-flow circuit. Previous in vitro studies have shown that in order to maximize the amount of drug delivered during high-flow oxygen therapy, the gas flow rate should be low and the nebulizer should be positioned immediately before or after the humidification chamber. This scenario was evaluated in Administration A.

The flow rate of 30 L/min for Administrations A and C was selected as being on the low end of the clinically relevant range, with the intention of maximizing drug delivery. Although oral inhalation in the presence of high-flow nasal oxygen therapy (Administration C) has been shown to be sub-optimal, this condition would be the most generalizable because it can be performed regardless of limitations to incorporating a nebulizer device in the various high-flow systems that are available and used in clinical settings worldwide.

The reference condition of oral inhalation in the presence of low-flow nasal oxygen therapy (Administration B) was selected on the basis of prior experience in a Phase 2 study in COVID-19. The majority of subjects in that study received nezulcitinib in this condition. Furthermore, the plasma concentrations of nezulcitinib in this condition were found to be similar to those in healthy subjects when nezulcitinib was administered by oral inhalation in the absence of supplemental oxygen therapy.

### Pharmacokinetic Sample Collection

Serial blood samples were collected prior to and until 48 hours following dosing on Day 1 of each period to determine the concentration of nezulcitinib in plasma.

### Pharmacokinetic Parameters and Analysis

The following non-compartmental PK parameters were calculated for nezulcitinib, as appropriate:

### Plasma:

AUC0-t: The area under the concentration time curve, from time 0 to the last observed non-zero concentration, as calculated by the linear trapezoidal method.
AUC0-24: The area under the concentration time curve, from time 0 to the 24 hour time point, as calculated by the linear trapezoidal method.
AUC0-inf: The area under the concentration time curve from time 0 extrapolated to infinity. AUC0-inf was calculated as the sum of AUC0-t plus the ratio of the last measurable plasma concentration to the elimination rate constant.
AUC % extrap: Percent of AUC0-inf extrapolated, represented as (1−AUC0-t/AUC0-inf)*100.
CL/F: Apparent total plasma clearance after oral (extravascular) administration, calculated as Dose/AUC0-inf.
Cmax: Maximum observed concentration.
Tmax: Time to reach Cmax. If the maximum value occurs at more than one time point, Tmax is defined as the first time point with this value.
Kel: Apparent first order terminal elimination rate constant calculated from a semi-log plot of the plasma concentration versus time curve. The parameter was calculated by linear least-squares regression analysis using the maximum number of points in the terminal log-linear phase (e.g., three or more non-zero plasma concentrations).
t_{1/2}: Apparent first-order terminal elimination half-life was calculated as 0.693/Kel.
Vz/F: Apparent volume of distribution during the terminal elimination phase after oral (extravascular) administration, calculated as Dose/(AUC0-inf*Kel).

The results for the non-compartmental PK parameter calculations for the various nezulcitinib administrations are presented in Table 2.

TABLE 2
Summary of Plasma Nezulcitinib Pharmacokinetic Parameters Following a Single Inhaled Dose

| Pharmacokinetic | Administration | Administration | Administration | Administration | Administration |
|---|---|---|---|---|---|
| Parameters | A | B | C | D1 | D2 |
| AUC0-24 | 7.060 ± | 49.11 ± | 49.91 ± | 47.88 ± | 141.4 ± |
| (ng*hr/mL) | 6.0242 [n = 14] | 25.304 [n = 14] | 32.502 [n = 14] | 26.219 [n = 7] | 110.81 [n = 6] |
| AUC0-t | 7.439 ± | 52.37 ± | 53.06 ± | 50.83 ± | 148.8 ± |
| (ng*hr/mL) | 6.6781 [n = 14] | 27.118 [n = 14] | 34.412 [n = 14] | 28.186 [n = 7] | 115.70 [n = 6] |
| DN AUC0-t | 2.480 ± | 17.46 ± | 17.69 ± | 16.94 ± | 16.53 ± |
| ([ng*hr/mL]/mg) | 2.2260 [n = 14] | 9.0393 [n = 14] | 11.471 [n = 14] | 9.3953 [n = 7] | 12.855 [n = 6] |
| AUC0-inf | 7.759 ± | 53.99 ± | 54.72 ± | 52.32 ± | 152.0 ± |
| (ng*hr/mL) | 6.9310 [n = 14] | 27.969 [n = 14] | 35.384 [n = 14] | 29.110 [n = 7] | 117.28 [n = 6] |
| DN AUC0-inf | 2.586 ± | 18.00 ± | 18.24 ± | 17.44 ± | 16.89 ± |
| ([ng*hr/mL]/mg) | 2.3103 [n = 14] | 9.3231 [n = 14] | 11.795 [n = 14] | 9.7035 [n = 7] | 13.031 [n = 6] |
| AUC % extrap | 4.451 ± | 3.152 ± | 3.898 ± | 2.813 ± | 2.727 ± |
| (%) | 1.8688 [n = 14] | 1.0247 [n = 14] | 3.5607 [n = 14] | 1.1206 [n = 7] | 1.1150 [n = 6] |
| Cmax (ng/mL) | 1.683 ± | 13.98 ± | 14.17 ± | 13.95 ± | 43.72 ± |
| | 1.0865 [n = 14] | 7.2429 [n = 14] | 8.4614 [n = 14] | 7.6933 [n = 7] | 37.338 [n = 6] |
| DN Cmax | 0.5610 ± | 4.658 ± | 4.725 ± | 4.649 ± | 4.858 ± |
| ([ng/mL]/mg) | 0.36215 [n = 14] | 2.4143 [n = 14] | 2.8205 [n = 14] | 2.5644 [n = 7] | 4.1486 [n = 6] |
| Tmax (hr) | 1.000 (1.00, | 1.000 (0.50, | 1.000 (1.00, | 1.000 (0.50, | 1.000 (1.00, |
| | 1.00) [n = 14] | 2.00) [n = 14] | 2.00) [n = 14] | 2.00) [n = 7] | 1.00) [n = 6] |
| Kel (1/hr) | 0.07122 ± | 0.07667 ± | 0.04804 ± | 0.06152 ± | 0.05057 ± |
| | 0.015970 [n = 14] | 0.096690 [n = 14] | 0.0081419 [n = 14] | 0.023529 [n = 7] | 0.0084453 [n = 6] |
| t½ (hr) | 10.179 ± | 12.889 ± | 15.076 ± | 12.336 ± | 14.058 ± |
| | 2.1842 [n = 14] | 3.4718 [n = 14] | 4.2716 [n = 14] | 3.4808 [n = 7] | 2.5336 [n = 6] |
| CL/F (L/hr) | 526.8 ± | 287.0 ± | 128.9 ± | 146.9 ± | 312.3 ± |
| | 216.71 [n = 14] | 832.49 [n = 14] | 210.97 [n = 14] | 240.87 [n = 7] | 611.78 [n = 6] |
| Vz/F (L) | 7400 ± | 1766 ± | 3921 ± | 1783 ± | 7313 ± |
| | 3018.9 [n = 14] | 1899.6 [n = 14] | 9194.6 [n = 14] | 1955.3 [n = 7] | 14752 [n = 6] |

Tmax are presented as median (min, max).
All other parameters are presented as arithmetic mean (±SD).

No value for Kel, AUC0-inf, AUC % extrap, CL/F, Vz/F, or t½ was reported for cases that did not exhibit a terminal log linear phase in the concentration time profile.

No PK parameters were calculated for participants with fewer than 3 consecutive time points with detectable concentrations.

PK parameters were summarized by administration using descriptive statistics.

An analysis of variance (ANOVA) was performed on the natural log (ln) transformed AUC0-t, AUC0-24, AUC0-inf, and Cmax, using the appropriate statistical procedure.

Twelve (12) participants were estimated to provide 90% power to detect a 2-fold difference between geometric means of the PK parameter AUC0-inf of tests (A, C, or D) to reference administration (B). Therefore, similarity was concluded if the 90% confidence intervals (CIs) for the ratios of geometric least-squares mean (LSM) of AUC0-t, AUC0-inf, and Cmax of nezulcitinib fell within 0.50 and 2.00.

### Safety Assessments

Safety was monitored through physical examinations including, at a minimum, examination of respiratory, cardiovascular, and gastrointestinal systems, with the option for further focused examination of additional systems as necessary based on reported symptoms/AEs, vital signs, 12-lead safety ECGs, spirometry, body weight monitoring, pulse oximetry, clinical laboratory tests (including, but not limited to, hematology, clinical chemistry profile, coagulation, and urinalysis), and AEs.

### Safety Analysis

The following analyses were performed; however, no formal inferential statistics were done on safety assessments.

Descriptive statistics were calculated for quantitative safety data and frequency counts were compiled for classification of qualitative safety data.

### Adverse Events

AEs were coded using the most current version of Medical Dictionary for Regulatory Activities (MedDRA®).

A by-participant AE data listing, including verbatim term, preferred term, system organ class (SOC), administration, severity, and relationship to drug, was provided.

Summaries of administration-emergent adverse events (AEAEs) were presented by SOC, preferred term, and the frequency and percentage of participants reporting each observed event. The frequency of participants who experience AEAEs was summarized overall and by study part and administration. AEs were also summarized by relationship to administration (study drug) and severity.

### Medical History and Physical Examination

Medical histories were listed by participant.

Clinically significant changes in physical examinations were captured as AEs.

### Clinical Laboratory Results, Electrocardiograms, Spirometry, Pulse Oximetry, and Vital Signs Measurements

All clinical laboratory results, 12-lead safety ECGs, spirometry measurements, pulse oximetry, vital signs measurements, and their change from baseline, were summarized by study part, administration, and time point of collection.

### Concomitant Medications

Concomitant medications were coded using the most current World Health Organization (WHO) drug dictionary and listed by participant.

### Study Design

This was a Phase 1, open-label, fixed-sequence, 4-period study to evaluate the PK and safety of single inhaled doses of nezulcitinib administered under different supplemental oxygenation scenarios.

Fourteen healthy, adult male and female participants were enrolled. Screening of participants occurred within 28 days prior to the first dosing.

On Day 1 of each period, participants received a single inhaled dose of nezulcitinib under one of the supplemental oxygenation scenarios. Blood samples for the assessment of plasma nezulcitinib concentrations were collected prior to and through 48 hours following each nezulcitinib administration. There was a washout period of at least 5 days between each nezulcitinib dose.

Safety (i.e., physical examinations, vital signs, 12-lead safety ECGs, spirometry, clinical laboratory tests, and AEs) were assessed throughout the study; blood, urine, and expired air samples were collected for safety assessments. Blood oxygenation levels were monitored via pulse oximetry prior to and during administration of supplemental oxygen. Discontinued participants were not replaced.

### Residency and Follow-Up

Participants were admitted to the Clinical Research Unit (CRU) on Day −1 of each study period and remained in residence through Hour 48 on Day 3 of each study period following completion of study assessments. Participants were admitted earlier than Day −1 for COVID-19 testing not related to study protocol as per CRU requirements. A participant may have been required to remain at the CRU for longer at the discretion of the Investigator or designee. As per site preference, participants were confined throughout the study (i.e., washout period[s]).

All participants who received at least one dose of nezulcitinib (including participants who terminate the study early) returned to the CRU 7 (±2) days after the last nezulcitinib administration for follow-up procedures, and to determine if any AEs had occurred since the last study visit.

The end of study was defined as the date of the last scheduled study procedure.

### Study Endpoints

Study endpoints for Part A were: AUC0-t, AUC0-24, AUC0-inf, AUC % extrap, Cmax, Tmax, CL/F, Vz/F, Kel, and t½ for nezulcitinib. Similarity was concluded if the 90% confidence intervals for the ratios of geometric least-squares means of AUC0-t, AUC0-inf, and Cmax of nezulcitinib fall within 0.50 and 2.00.

Study endpoints for Part B were: administration emergent adverse events, physical examinations, vital signs, 12-lead safety ECGs, spirometry, pulse oximetry, and clinical laboratory tests.

### Results

All 14 subjects completed 3 periods (treatments), with 13 subjects completing all 4 periods (1 subject withdrew consent after the 3rd period for personal scheduling reasons). 7 subjects completed Treatment D1 (3 mg dose of nezulcitinib via nebulized oral inhalation, with high-flow nasal cannula at a rate of 50 L/min) and 6 subjects completed Treatment D2 (9 mg dose of nezulcitinib via nebulized oral inhalation, with high-flow nasal cannula at a rate of 50 L/min).

Adverse events occurred across 2 subjects (14.3%): 2 episodes of nasal congestion in 1 subject, and 1 episode of epistaxis in another. All adverse events were of mild severity, and were considered not related to the study drug. Further, all adverse events resolved during the course of the study, and no adverse events led to discontinuation of dosing or withdrawal from the study.

There were also no clinically relevant changes seen in other subject safety assessments: safety laboratory measures (clinical chemistry and hematologic parameters), safety electrocardiograms, vital signs assessments, pulse oximetry, or spirometry.

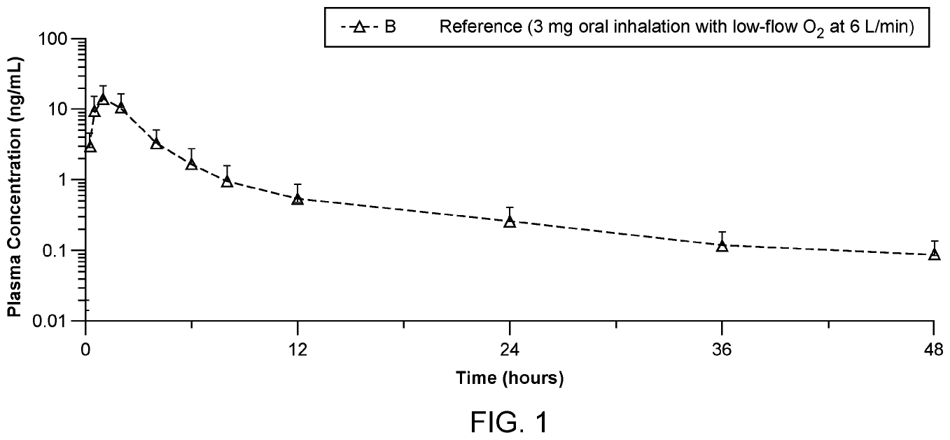

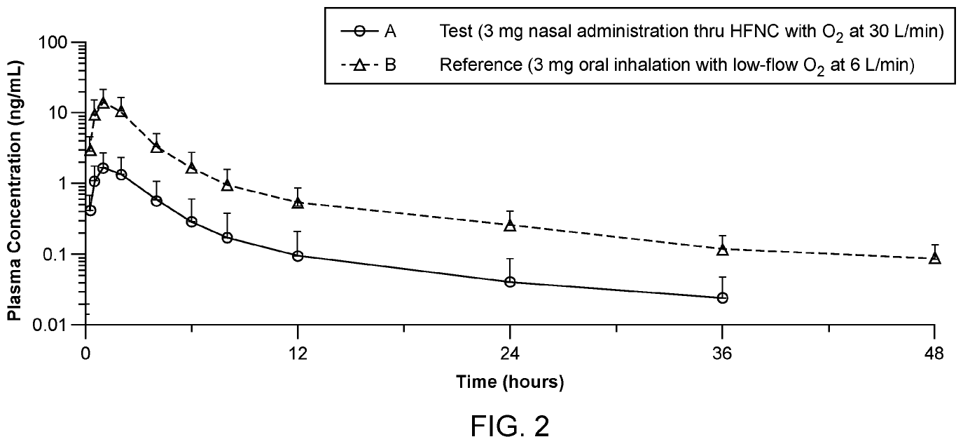

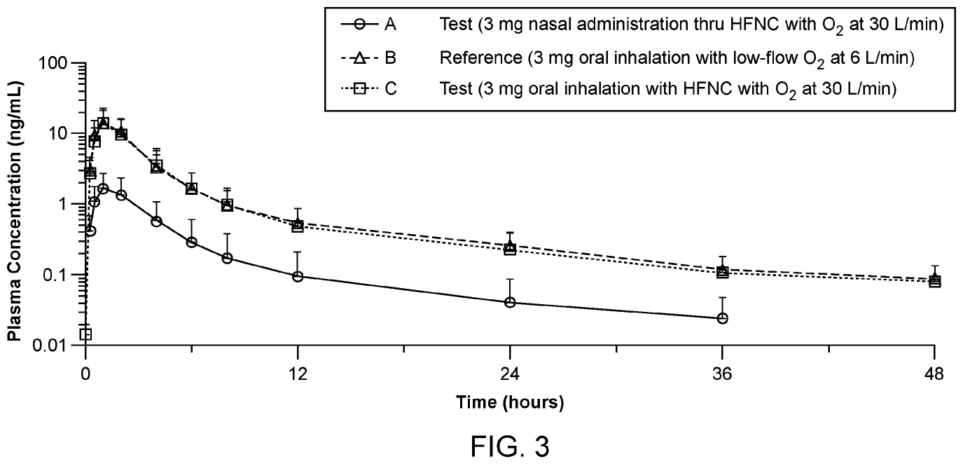

Plasma concentrations of nezulcitinib in subjects as a function of time are shown in FIGS. 1-6. Surprisingly, administration of 3 mg of nezulcitinib via nebulized oral inhalation in the presence of nasal high-flow oxygen at a flow rate of 30 L/min (Administration C, test) provided sustained increased plasma concentrations when compared to nasal administration of 3 mg of nezulcitinib through the high-flow circuit at a flow rate of 30 L/min (Administration A, test), and results in similar plasma concentrations when compared to nebulized oral inhalation of 3 mg of nezulcitinib in the presence of nasal low-flow oxygen at a flow rate of 6 L/min (Administration B, reference), as can be seen in FIG. 3.

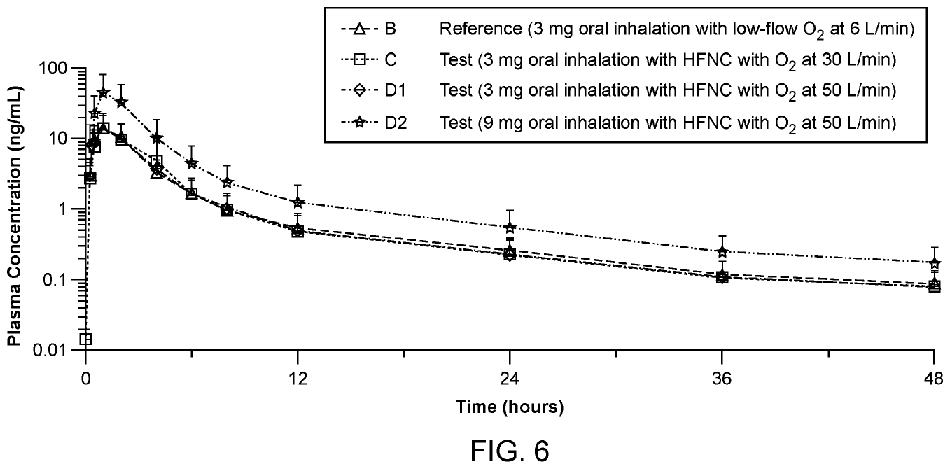

Increasing the flow rate through the nasal high-flow oxygen cannula from 30 L/min (Administration C, test) to 50 L/min (Administration D1, test) did not impact the plasma concentrations associated with administration of 3 mg of nezulcitinib via nebulized oral inhalation. Further, at a nasal high-flow oxygen cannula flow rate of 50 L/min, increasing the nebulized oral inhalation dose from 3 mg of nezulcitinib (Administration D1, test) to 9 mg of nezulcitinib (Administration D2, test) resulted in approximately a dose-proportional increase in plasma concentration, as can be seen in FIG. 6.

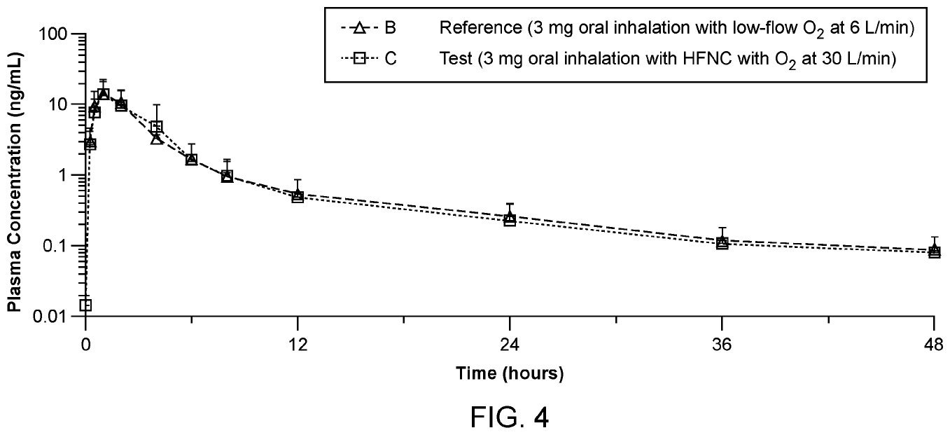

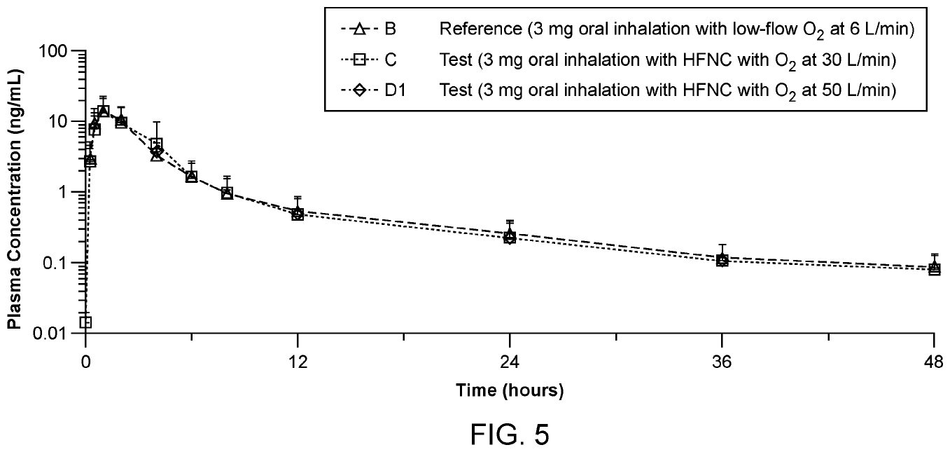

As can be seen in FIG. 4, subjects administered a 3 mg dose of nezulcitinib via oral nebulized inhalation while also receiving high-flow supplemental oxygen at a rate of 30 L/min via a high-flow nasal cannula (Administration C, test) displayed a comparable plasma concentration of active agent when compared to subjects receiving a similar 3 mg dose of nezulcitinib via oral nebulized inhalation while only receiving low-flow supplemental oxygen at a rate of 6 L/min (Administration B, reference), indicating that administration of nezulcitinib via oral nebulized inhalation can provide adequate active agent to the subject's respiratory system regardless of rate of nasal supplemental oxygen being administered in parallel. This result is further supported in FIG. 5, with subjects being administered a 3 mg dose of nezulcitinib via oral nebulized inhalation while also receiving high-flow supplemental oxygen at a rate of 50 L/min via a high-flow nasal cannula (Administration D1, test) displaying similar plasma concentration of nezulcitinib to subjects receiving Administration B or Administration C.

In summary, administration of a pharmaceutical composition comprising nezulcitinib, or a pharmaceutically acceptable salt thereof, to a subject by oral nebulized inhalation while the subject is receiving high-flow oxygen through a nasal cannula at a rate greater than or equal to about 30 L/min was found to be unexpectedly superior to administration through the in-line high-flow circuit, contrary to current recommendations in the art. Moreover, administration by oral nebulized inhalation to subjects receiving high-flow and low-flow oxygen resulted surprisingly in similar exposure. Thus, it has now been discovered that administration of a pharmaceutical composition comprising nezulcitinib, or a pharmaceutically acceptable salt thereof, to a subject in need thereof through the high-flow nasal circuit is unnecessary and inferior to oral nebulized inhalation; and the flow of oxygen does not need to be reduced when the pharmaceutical composition is being administered to the subject.

### Example 2—Administration of a Nezulcitinib Inhalation Solution to a Subject Receiving Oxygen Via a High-Flow Nasal Cannula (HFNC)

The nezulcitinib solution for inhalation employed in this example is a clear, colorless-to-yellow solution, consisting of 3 mg of nezulcitinib in a sterile aqueous solution, containing 10 mM citrate buffer, with 150 mM sodium chloride, at pH 4.0. The nezulcitinib inhalation solution is provided in unit dose, low-density polyethylene (LDPE) vials, manufactured by Blow-Fill-Seal (BFS) technology and individually packaged in sealed foil laminate pouches. Each pouch contains one unit vial containing 1 mL of a 3 mg/mL concentration nezulcitinib inhalation solution. The nezulcitinib inhalation solution pouches should be stored at a temperature between 2° and 8° C.

The nezulcitinib inhalation solution can be administered to subjects receiving supplemental oxygen via HFNC by oral inhalation using an Aerogen Solo+Ultra nebulizer system. The following representative procedure can be followed for administration of the nezulcitinib inhalation solution.

1. If the oxygen flow rate is greater than 50 L/min, turn flow rate down to 50 L/min, adjusting FiO2 if needed and allow subject to acclimate to the adjustment while preparing the device.

2. Ensure all needed supplies are available including:
(i) Aerogen Controller (can be used for multiple subjects)
(ii) Aerogen Solo nebulizer (used for up to one week for each subject)
(iii) New Aerogen Ultra handset and filter (single-use, disposed daily)
(iv) 3 vials of nezulcitinib inhalation solution (3 mg nezulcitinib per vial for a total dose of 9 mg)

3. Confirm the subject ID number written on the pouch label matches the subject ID for the subject to receive the dose and record the lot number, dose, and dose volume in the subject's source records.

4. Assemble the Aerogen Solo Nebulizer system according to the product instructions, leaving the medication reservoir open, and connect it to a clean Aerogen controller.

5. Open each pouch of nezulcitinib inhalation solution and remove the vials. Open and remove one vial and squeeze the entire contents into the reservoir cup of the Aerogen Solo; the subject will then nebulize the contents of the vial (approximately 3 minutes), and this will be repeated 3 times for each of 3 vials, separated by a 1-minute break in-between each vial (see Step 10 below).

Important: To avoid irritation due to inhaling aerosol when it is too cold, do not use nezulcitinib inhalation solution straight from the refrigerator. Warm in the hands if required.

6. Close the nebulizer reservoir.

7. Do not tilt the nebulizer handset more than 45 degrees (up, down, or sideways) after putting the nezulcitinib inhalation solution into the reservoir.

8. Check that all parts of the nebulizer are connected tightly and that the reservoir is closed.

9. Pass the Aerogen Ultra handset to the subject, and if possible, ensure they are sitting upright (greater than 45 degrees), with elbows resting on a tray or solid support, and holding the device in the proper orientation. If subject is unable to hold nebulizer device for dosing, staff may assist. Instruct the subject to make a seal with their lips around the mouthpiece and to practice breathing normally in and out through the mouthpiece of the device for a few moments, to allow acclimation of oral breathing in the presence of nasal high-flow oxygen. It is important that the subject can breathe comfortably through the mouth during the dosing procedure.

10. Press off/on button to begin administration of study drug. Instruct the subject to breathe normally in and out through the mouthpiece during dosing. The start and end time of inhalation should be recorded in the subject source notes. Nebulization time for each 1 mL vial should take approximately 3 minutes. With a 1-minute pause between vials, dosing should be complete within approximately 15 minutes after the start of the nebulization. If extended breaks are necessary for re-oxygenation, dosing time will increase. The subject will receive three 1 mL vials of nezulcitinib inhalation solution, for a total dose of 9 mg. The contents of each vial should be squeezed into the nebulizer (Aerogen Solo) and administered as follows:
(a) Proceed to nebulize the complete 1 mL volume for vial #1 (approximately 3 mins). Note: Foaming may occur in the reservoir cup of the Aerogen Solo during dosing. Medication will continue to be delivered despite foaming. Stop the controller when the nebulizer cup is empty and no more mist is visible coming out of the nebulizer (approximately 3 minutes from the start of dosing for each vial).
(b) The nebulizer should be turned off and the subject should then remove the nebulizer mouthpiece from their mouth and pause for approximately 1 minute or more, as needed to rest and re-oxygenate.
   During the pause, the subject should be coached to spit or swallow if saliva build-up occurred during the oral inhalation procedure. Also during the pause, another 1 mL vial should be added to the nebulizer.
(c) After approximately 1 minute, the subject should place the nebulizer mouthpiece back into their mouth, and the nebulizer should be activated to resume dosing, until the full contents of the vial are administered (approximately 3 minutes).
(d) Once the second vial is completed, complete steps (b) and (c) again, to administer the third vial.

11. Once the nezulcitinib inhalation solution administration is complete, the Aerogen Solo nebulizer should be removed from the Ultra handset and stored for re-use with the same subject for up to 7 days. The Ultra handset and filter should be discarded after each use.

12. The Aerogen USB controller can be used for multiple subjects, and should be cleaned between each administration by wiping clean with an alcohol based disinfectant wipe or a quaternary ammonium compound based disinfectant wipe. Do not (i) spray liquid directly onto the Aerogen USB controller; (ii) immerse the Aerogen USB controller in liquid; or (iii) sterilize (autoclave) the Aerogen USB controller.

While the present disclosure has been described with reference to specific aspects or embodiments thereof, it will be understood by those of ordinary skilled in the art that various changes can be made or equivalents can be substituted without departing from the true spirit and scope of the disclosure. Additionally, to the extent permitted by applicable patent statutes and regulations, all publications, patents and patent applications cited herein are hereby incorporated by reference in their entirety to the same extent as if each document had been individually incorporated by reference herein.

What is claimed is:

1. A method for treating a respiratory disorder in a subject in need thereof when the subject is receiving high-flow oxygen through a nasal cannula, the method comprising administering a pharmaceutical composition comprising nezulcitinib, or a pharmaceutically acceptable salt thereof, to the subject by oral nebulized inhalation while the subject is receiving high-flow oxygen through a nasal cannula at a rate greater than or equal to about 30 L/min.

2. The method of claim 1, wherein the subject is receiving high-flow oxygen through a nasal cannula at a rate of about 30 L/min to about 50 mL/min.

3. The method of claim 1 or 2, wherein the pharmaceutical composition comprises about 1 mg to about 10 mg of nezulcitinib or a pharmaceutically acceptable salt thereof.

4. The method of any one of claims 1 to 3, wherein the pharmaceutical composition comprises about 3 mg of nezulcitinib or a pharmaceutically acceptable salt thereof.

5. The method of any one of claims 1 to 4, wherein the pharmaceutical composition comprises about 3 mg/l mL (free base equivalent) of nezulcitinib or a pharmaceutically acceptable salt thereof.

6. The method of any one of claims 1 to 5, wherein the pharmaceutical composition has a pH in the range of about 3.5 to about 4.5.

7. The method of any one of claims 1 to 6, wherein the pharmaceutical composition has a pH of about 4.0.

8. The method of any one of claims 1 to 7, wherein the pharmaceutical composition further comprises a buffering agent.

9. The method of claim 8, wherein the buffering agent is citric acid.

10. The method of any one of claims 1 to 9, wherein the pharmaceutical composition further comprises a tonicity agent.

11. The method of claim 10, wherein the tonicity agent is sodium chloride.

12. The method of any one of claims 1 to 11, wherein the pharmaceutical composition is administered once a day.

13. The method of any one of claims 1 to 12, wherein the respiratory disorder is selected from the group consisting of a viral infection, lung transplant rejection, asthma, chronic obstructive pulmonary disease, cystic fibrosis, pneumonitis, idiopathic pulmonary fibrosis, acute lung injury, acute respiratory distress syndrome, bronchitis, emphysema, bronchiolitis obliterans, sarcoidosis, an eosinophilic disease, a helminthic infection, pulmonary arterial hypertension, lymphangioleiomyomatosis, bronchiectasis, an infiltrative pulmonary disease, drug-induced pneumonitis, fungal induced pneumonitis, allergic bronchopulmonary aspergillosis, hypersensitivity pneumonitis, eosinophilic granulomatosis with polyangiitis, idiopathic acute eosinophilic pneumonia, idiopathic chronic eosinophilic pneumonia, hypereosinophilic syndrome, Löffler's syndrome, bronchiolitis obliterans organizing pneumonia, lung graft-versus-host disease, and immune-checkpoint-inhibitor induced pneumonitis.

14. The method of any one of claims 1 to 13, wherein the respiratory disorder is a coronavirus infection.

15. The method of claim 14, wherein the coronavirus infection is caused by SARS-CoV-1, SARS-CoV-2, or MERS-CoV.

16. The method of claim 14, wherein the coronavirus infection is caused by SARS-CoV-2.

17. The method of any one of claims 1 to 16, wherein the subject has acute lung injury associated with COVID-19.

18. The method of any one of claims 1 to 17, wherein the subject has a baseline level of C-reactive protein less than or equal to about 150 mg/L.

19. The method of any one of claims 1 to 13, wherein the respiratory disorder is asthma or chronic obstructive pulmonary disease.

20. The method of any one of claims 1 to 13, wherein the respiratory disorder is lung transplant rejection.

21. A method for treating a SARS-CoV-2 lung infection in a subject in need thereof when the subject is receiving high-flow oxygen through a nasal cannula, the method comprising administering a pharmaceutical composition comprising nezulcitinib, or a pharmaceutically acceptable salt thereof, to the subject by oral nebulized inhalation while the subject is receiving high-flow oxygen through a nasal cannula at a rate greater than or equal to about 30 L/min.

22. The method of claim 21, wherein the subject is receiving high-flow oxygen through a nasal cannula at a rate of about 30 L/min to about 50 mL/min.

23. The method of claim 21 or 22, wherein the pharmaceutical composition comprises about 1 mg to about 10 mg of nezulcitinib or a pharmaceutically acceptable salt thereof.

24. The method of any one of claims 21 to 23, wherein the pharmaceutical composition comprises about 3 mg of nezulcitinib or a pharmaceutically acceptable salt thereof.

25. The method of any one of claims 21 to 24, wherein the pharmaceutical composition comprises about 3 mg/l mL (free base equivalent) of nezulcitinib or a pharmaceutically acceptable salt thereof.

26. The method of any one of claims 21 to 25, wherein the pharmaceutical composition has a pH in the range of about 3.5 to about 4.5.

27. The method of any one of claims 21 to 26, wherein the pharmaceutical composition has a pH of about 4.0.

28. The method of any one of claims 21 to 27, wherein the pharmaceutical composition further comprises a buffering agent.

29. The method of claim 28, wherein the buffering agent is citric acid.

30. The method of any one of claims 21 to 29, wherein the pharmaceutical composition further comprises a tonicity agent.

31. The method of claim 30, wherein the tonicity agent is sodium chloride.

32. The method of any one of claims 21 to 31, wherein the pharmaceutical composition is administered once a day.

33. The method of any one of claims 21 to 32, wherein the subject has acute lung injury associated with COVID-19.

34. The method of any one of claims 21 to 33, wherein the subject has a baseline level of C-reactive protein less than or equal to about 150 mg/L.

35. A method for treating a lung disease, disorder, or condition in a subject in need thereof when the subject is receiving high-flow oxygen through a nasal cannula, the method comprising administering a pharmaceutical composition comprising a therapeutic agent, or a pharmaceutically acceptable salt thereof, to the subject by oral nebulized inhalation while the subject is receiving high-flow oxygen through a nasal cannula at a rate greater than or equal to about 30 L/min.

36. The method of claim 35, wherein the subject is receiving high-flow oxygen through a nasal cannula at a rate of about 30 L/min to about 50 mL/min.

37. The method of claim 35 or 36, wherein the therapeutic agent is a JAK inhibitor.

38. The method of any one of claims 35-37, wherein the therapeutic agent is nezulcitinib, and the pharmaceutical composition comprises about 1 mg to about 10 mg of nezulcitinib or a pharmaceutically acceptable salt thereof.

39. The method of any one of claims 35-38, wherein the lung disease, disorder, or condition is a respiratory disorder.

40. The method of claim 39, wherein the respiratory disorder is a viral infection, lung transplant rejection, asthma, chronic obstructive pulmonary disease, cystic fibrosis, pneumonitis, idiopathic pulmonary fibrosis, acute lung injury, acute respiratory distress syndrome, bronchitis, emphysema, bronchiolitis obliterans, sarcoidosis, an eosinophilic disease, a helminthic infection, pulmonary arterial hypertension, lymphangioleiomyomatosis, bronchiectasis, an infiltrative pulmonary disease, drug-induced pneumonitis, fungal induced pneumonitis, allergic bronchopulmonary aspergillosis, hypersensitivity pneumonitis, eosinophilic granulomatosis with polyangiitis, idiopathic acute eosinophilic pneumonia, idiopathic chronic eosinophilic pneumonia, hypereosinophilic syndrome, Löffler's syndrome, bronchiolitis obliterans organizing pneumonia, lung graft-versus-host disease, and immune-checkpoint-inhibitor induced pneumonitis.

41. The method of any one of the preceding claims, wherein the nebulizer is a mesh nebulizer.

42. The method of any one of the preceding claims, wherein delivered atomized particles are comprised of particles substantially having a mean diameter of less than about 10 μm.
